(19) United States
(12) Patent Application Publication
(10) Pub. No.: US 2026/0201043 A1
(43) Pub. Date: Jul. 16, 2026

(54) MONOVALENT ANTIBODIES SPECIFIC FOR GALECTINS AND USES THEREOF

(71) Applicant: INSTITUT NATIONAL DE LA RECHERCHE SCIENTIFIQUE, Québec (CA)

(72) Inventors: Yves ST-PIERRE, Canmore (CA); Nicolas DOUCET, Laval (CA); David CHATENET, Lorraine (CA)

(21) Appl. No.: 19/128,089

(22) PCT Filed: Nov. 7, 2023
(86) PCT No.: PCT/CA2023/051484

§ 371 Date: May 7, 2025

Related U.S. Application Data
(60) Provisional application No. 63/382,803, filed on Nov. 8, 2022.

(51) Int. Cl. C07K 16/28; A61K 47/68; A61K 51/10

(52) U.S. Cl. CPC C07K 16/2851; A61K 47/6849; A61K 51/1027; C07K 2317/569

(57) ABSTRACT

Monovalent antibodies such as single-domain antibodies (sdAbs) that are specific for galectin-13/16 or galectin-9 are described. These monovalent antibodies are able to interfere with the activity of galectin-13/16 or galectin-9, and thus may be used for the treatment of diseases associated with dysregulated galectin-13/16 or galectin-9 expression and/or activity, such as certain types of cancers, as well as conditions associated with inflammation.

### CROSS-REFERENCE TO RELATED APPLICATIONS

The present application claims the benefit of U.S. provisional patent application No. 63/382,803, filed on Nov. 8, 2022, which is incorporated herein by reference.

### TECHNICAL FIELD

The present disclosure generally relates to galectins, and more specifically to the detection and/or modulation of activity of galectin-9 (GAL-9), galectin-13 (GAL-13) and/or galectin-16 (GAL-16).

### BACKGROUND ART

In biology, carbohydrates are defined as organic compounds composed of carbon, hydrogen, and oxygen organized into ring structures. Analyzing these structures and their functions has led to a new field of biology called “glycobiology.” In the biomedical sciences, glycobiology is rapidly emerging as an integral part of complex biological processes. Evidence suggesting that the interactions between lectins and their ligands play a major role in the different steps of cancer progression has accumulated at a rapid pace and has gained the attention of several oncologists [reviewed by Pinho and Reis, 2015]. This is particularly true for galectin family members because changes in their expression levels correlate with alterations in cancer cell growth, apoptosis, and cell-cell and cell-matrix interactions [reviewed by Liu and Rabinovich, 2005].

Galectins (GAL) are multifunctional proteins that belong to the animal lectin family. All galectins share similar binding affinities to β-galactosides and display some sequence and structural similarities among their carbohydrate-recognition domains (CRDs) [Barondes et al., 1994]. Galectins can be found in the cytoplasm, the nucleus, or can be secreted by the cell, a mechanism which occurs via a non-classical secretory pathway. The distribution of galectins is tissue specific, and their expression is developmentally regulated [Cummings and Liu, 2009]. In mammals, 19 different members of this family have been identified, with 13 of them being expressed in humans. GAL-5, -6, -11, -15, -16, -19, and -20 are not found in humans. Galectins are divided into three sub-groups according to their structure: prototypic galectins containing one CRD (GAL-1, -2, -5, -7, -10, -11, -13, -14, -15, -16, -17, -19, and -20), tandem-repeat galectins containing two covalently linked CRDs (GAL-4, -6, -8, -9 and -12) and chimera-type galectins containing multiple CRDs linked by their amino-terminal domain (GAL-3).

Although galectins are involved in various physiological processes, they are best known for their immunoregulatory roles when released either passively from dead cells or actively via non-classical secretion pathways [Liu et al., 2008; Rabinovich and Toscano, 2009]. This was documented first in a landmark paper published by the group of Linda Baum showing that galectin-1 (GAL-1) can induce apoptosis of activated T cells [Perillo et al., 1997]. The immunosuppressive role of galectins is also highlighted by studies showing that administration of recombinant prototypic GAL-1 prevents disease progression in patients with autoimmune disorders [Sunblad et al., 2017; Allo et al., 2020]. Since the initial discovery by the group of Linda Baum, the immunoregulatory role of galectins has been extended to most if not all members of the family. For example, several galectin family members, such as GAL-13, GAL-14, and GAL-16, contribute to the generation of an immune-privileged environment at the maternal-fetal interface [Than et al., 2014].

Expression of galectins undergoes strict mechanisms of regulation. This is not surprising given their important role in the regulation of the immune response. In cancer cells, however, the expression of galectins often reaches abnormally high levels, favoring an increase of galectin concentrations in the extracellular milieu [Grosset et al., 2016; Labrie et al., 2017]. Such accumulation of galectins creates systemic and local immunosuppressive microenvironments that promotes cancer progression and metastasis, which represents a significant obstacle to cancer immunotherapy [reviewed by Rodriguez et al., 2018 and more recently by Jin et al., 2021].

Given their important role in cancer and other diseases, considerable efforts have been directed towards the development of galectin inhibitors. Despite almost two decades of research, however, the development of effective and specific galectin-1 antagonists has met limited success [Blanchard et al., 2016]. In most cases, these inhibitors are high molecular weight, naturally occurring polysaccharides that are used to block the binding of extracellular galectins to carbohydrate structures on cell surface receptors. Yet, the greatest challenge to these glycan-binding site (GBS) targeting drugs is achieving high selectivity. This is a challenging task considering the striking structural similarity between the GBS of multiple homologous galectins [Cummings and Liu, 2009]. Such structural similarity also complicates the use of conventional blocking antibodies, a logical strategy to generate inhibitors that would specifically target de GBS of galectins. Moreover, the use of conventional antibodies for the treatment of solid tumors is a challenging task given their poor tissue penetration [Cruz and Kayser, 2019].

There is thus a need for the development of novel agents to detect and/or modulate the activity of galectins such as GAL-9, GAL-13 and GAL-16.

### SUMMARY

The present disclosure provides the following items 1 to 118:
1. A monovalent antibody that binds to human galectin-13 (GAL-13), wherein the monovalent antibody comprises one of the following combinations of complementarity determining regions (CDRs):
   a CDR1 comprising or consisting of an amino acid sequence having at least 70% identity with the sequence FGSNRST (SEQ ID NO:22); a CDR2 comprising or consisting of an amino acid sequence having at least 70% identity with the sequence SLSSDPP (SEQ ID NO:23); and a CDR3 comprising or consisting of an amino acid sequence having at least 70% identity with the sequence PAGMRGVME (SEQ ID NO:24);
   a CDR1 comprising or consisting of an amino acid sequence having at least 70%, 75%, 80%, 85% or 90% identity with the sequence STSRSSG (SEQ ID NO:25); a CDR2 comprising or consisting of an amino acid sequence having at least 70%, 75%, 80%, 85% or 90% identity with the sequence, YRGSGAR (SEQ ID NO:26); and a CDR3 comprising or consisting of an amino acid sequence having at least 80%, 85% or 90% identity with the sequence MWQRQIQPGTRPVMK (SEQ ID NO:27);
   a CDR1 comprising or consisting of an amino acid sequence having at least 70% identity with the sequence RGSRWYG (SEQ ID NO:28); a CDR2 comprising or consisting of an amino acid sequence having at least 70% identity with the sequence AQHSTRA (SEQ ID NO:29); and a CDR3 comprising or consisting of an amino acid sequence having at least 70% identity with the sequence GYMSKMGERKWK (SEQ ID NO:30);
   a CDR1 comprising or consisting of an amino acid sequence having at least 70% identity with the sequence SSYAGSA (SEQ ID NO:31); a CDR2 comprising or consisting of an amino acid sequence having at least 70% identity with the sequence SDPDTKA (SEQ ID NO:32); and a CDR3 comprising or consisting of an amino acid sequence having at least 70% identity with the sequence ISGQYPYAR (SEQ ID NO:33);
   a CDR1 comprising or consisting of an amino acid sequence having at least 70% identity with the sequence YTSNEDS (SEQ ID NO:34); a CDR2 comprising or consisting of an amino acid sequence having at least 70% identity with the sequence RGPSFRT (SEQ ID NO:35); and a CDR3 comprising or consisting of an amino acid sequence having at least 70% identity with the sequence IRLGRLYRYGDLTPKSAY (SEQ ID NO:36);
   a CDR1 comprising or consisting of an amino acid sequence having at least 70% identity with the sequence RTSSLTS (SEQ ID NO:37); a CDR2 comprising or consisting of an amino acid sequence having at least 70% identity with the sequence FSGSMFT (SEQ ID NO:38); and a CDR3 comprising or consisting of an amino acid sequence having at least 70% identity with the sequence DKWMHKPAN (SEQ ID NO:39);
   a CDR1 comprising or consisting of an amino acid sequence having at least 70% identity with the sequence RTWNSYT (SEQ ID NO:40); a CDR2 comprising or consisting of an amino acid sequence having at least 70% identity with the sequence GEPGGIR (SEQ ID NO:41); and a CDR3 comprising or consisting of an amino acid sequence having at least 70% identity with the sequence DRWPNKHDR (SEQ ID NO:42);
   a CDR1 comprising or consisting of an amino acid sequence having at least 70% identity with the sequence TTWKQET (SEQ ID NO:43); a CDR2 comprising or consisting of an amino acid sequence having at least 70% identity with the sequence RGPNYYP (SEQ ID NO:44); and a CDR3 comprising or consisting of an amino acid sequence having at least 70% identity with the sequence DEWMGSINARHGSGY (SEQ ID NO:45);
   a CDR1 comprising or consisting of an amino acid sequence having at least 70% identity with the sequence RGSALTT (SEQ ID NO:46); a CDR2 comprising or consisting of an amino acid sequence having at least 70% identity with the sequence GDTGLYV (SEQ ID NO:47); and a CDR3 comprising or consisting of an amino acid sequence having at least 70% identity with the sequence GKWLGGRQADTR (SEQ ID NO:48); or a CDR1 comprising or consisting of an amino acid sequence having at least 70% identity with the sequence RTSNQT (SEQ ID NO:49); a CDR2 comprising or consisting of an amino acid sequence having at least 70% identity with the sequence ARFDEYG (SEQ ID NO:50); and a CDR3 comprising or consisting of an amino acid sequence having at least 70% identity with the sequence LGWPQKSTWRAYTAE (SEQ ID NO:51).
2. The monovalent antibody of item 1, wherein said at least 70% identity is at least 90% identity.
3. The monovalent antibody of item 1 or 2, which comprises the following combinations of CDRs: a CDR1 comprising or consisting of the sequence FGSNRST (SEQ ID NO:22); a CDR2 comprising or consisting of the sequence SLSSDPP (SEQ ID NO:23); and a CDR3 comprising or consisting of the sequence PAGMRGVME (SEQ ID NO:24).
4. The monovalent antibody of item 1 or 2, which comprises the following combinations of CDRs: a CDR1 comprising or consisting of the sequence STSRSSG (SEQ ID NO:25); a CDR2 comprising or consisting of the sequence, YRGSGAR (SEQ ID NO:26); and a CDR3 comprising or consisting of the sequence MWQRQIQPGTRPVMK (SEQ ID NO:27).
5. The monovalent antibody of item 1 or 2, which comprises the following combinations of CDRs: a CDR1 comprising or consisting of the sequence RGSRWYG (SEQ ID NO:28); a CDR2 comprising or consisting of the sequence AQHSTRA (SEQ ID NO:29); and a CDR3 comprising or consisting of the sequence GYMSKMGERKWK (SEQ ID NO:30).
6. The monovalent antibody of item 1 or 2, which comprises the following combinations of CDRs: a CDR1 comprising or consisting of the sequence SSYAGSA (SEQ ID NO:31); a CDR2 comprising or consisting of the sequence SDPDTKA (SEQ ID NO:32); and a CDR3 comprising or consisting of the sequence ISGQYPYAR (SEQ ID NO:33).
7. The monovalent antibody of item 1 or 2, which comprises the following combinations of CDRs: a CDR1 comprising or consisting of the sequence YTSNEDS (SEQ ID NO:34); a CDR2 comprising or consisting of the sequence RGPSFRT (SEQ ID NO:35); and a CDR3 comprising or consisting of the sequence IRLGRLYRYGDLTPKSAY (SEQ ID NO:36).
8. The monovalent antibody of item 1 or 2, which comprises the following combinations of CDRs: a CDR1 comprising or consisting of the sequence RTSSLTS (SEQ ID NO:37); a CDR2 comprising or consisting of the sequence FSGSMFT (SEQ ID NO:38); and a CDR3 comprising or consisting of the sequence DKWMHKPAN (SEQ ID NO:39).
9. The monovalent antibody of item 1 or 2, which comprises the following combinations of CDRs: a CDR1 comprising or consisting of the sequence RTWNSYT (SEQ ID NO:40); a CDR2 comprising or consisting of the sequence GEPGGIR (SEQ ID NO:41); and a CDR3 comprising or consisting of the sequence DRWPNKHDR (SEQ ID NO:42).
10. The monovalent antibody of item 1 or 2, which comprises the following combinations of CDRs: a CDR1 comprising or consisting of the sequence TTWKQET (SEQ ID NO:43); a CDR2 comprising or consisting of the sequence RGPNYYP (SEQ ID NO:44); and a CDR3 comprising or consisting of the sequence DEWMGSINARHGSGY (SEQ ID NO:45).
11. The monovalent antibody of item 1 or 2, which comprises the following combinations of CDRs: a CDR1 comprising or consisting of the sequence RGSALTT (SEQ ID NO:46); a CDR2 comprising or consisting of the sequence GDTGLYV (SEQ ID NO:47); and a CDR3 comprising or consisting of the sequence GKWLGGRQADTR (SEQ ID NO:48).
12. The monovalent antibody of item 1 or 2, which comprises the following combinations of CDRs: a CDR1 comprising or consisting of the sequence RTSNQT (SEQ ID NO:49); a CDR2 comprising or consisting of the sequence ARFDEYG (SEQ ID NO:50); and a CDR3 comprising or consisting of the sequence LGWPQKSTWRAYTAE (SEQ ID NO:51).
13. The monovalent antibody of any one of items 1 to 12, wherein the monovalent antibody is a single-domain antibody.
14. The monovalent antibody of any one of items 1 to 13, which comprises:
   (i) a framework region (FR) 1 comprising an amino acid sequence having at least 50% identity with the sequence MAEVQLQASGGGFVQPGGSLRLSCAASG (SEQ ID NO:52) or IAEVQLQASGGGFVQPGGSLRLSCAASG (SEQ ID NO:68);
   (ii) a FR2 comprising an amino acid sequence having at least 50% identity with the sequence MGWFRQAPGKEREFVSAIS (SEQ ID NO:53);
   (iii) a FR3 comprising or consisting of an amino acid sequence having at least 50% identity with the sequence YYADSVKGRFTISRDNSKNTVYLQMNSLRAEDTATYYCA (SEQ ID NO: 54);
   (iv) a FR4 comprising an amino acid sequence having at least 50% identity with the sequence YWGQGTQVTVSS (SEQ ID NO:55) or YWGHGTQVTVSS (SEQ ID NO:69); or
   (v) any combination of (i) to (iv).
15. The monovalent antibody of item 14, which comprises:
   (i) a FR1 comprising an amino acid sequence having at least 90% identity with the sequence MAEVQLQASGGGFVQPGGSLRLSCAASG (SEQ ID NO:52) or IAEVQLQASGGGFVQPGGSLRLSCAASG (SEQ ID NO:68);
   (ii) a FR2 comprising an amino acid sequence having at least 90% identity with the sequence MGWFRQAPGKEREFVSAIS (SEQ ID NO:53);
   (iii) a FR3 comprising or consisting of an amino acid sequence having at least 90% identity with the sequence YYADSVKGRFTISRDNSKNTVYLQMNSLRAEDTATYYCA (SEQ ID NO: 54);
   (iv) a FR4 comprising an amino acid sequence having at least 90% identity with the sequence YWGQGTQVTVSS (SEQ ID NO:55) or YWGHGTQVTVSS (SEQ ID NO:69); or
   (v) any combination of (i) to (iv).
16. The monovalent antibody of item 15, which comprises:
   (i) a FR1 comprising the sequence MAEVQLQASGGGFVQPGGSLRLSCAASG (SEQ ID NO: 52) or IAEVQLQASGGGFVQPGGSLRLSCAASG (SEQ ID NO:68);
   (ii) a FR2 comprising the sequence MGWFRQAPGKEREFVSAIS (SEQ ID NO:53);
   (iii) a FR3 comprising the sequence YYADSVKGRFTISRDNSKNTVYLQMNSLRAEDTATYYCA (SEQ ID NO:54);
   (iv) a FR4 comprising the sequence YWGQGTQVTVSS (SEQ ID NO:55) or YWGHGTQVTVSS (SEQ ID NO:69); or
   (v) any combination of (i) to (iv).
17. The monovalent antibody of any one of items 1 to 16, comprising an amino acid sequence having at least 80% identity with one of the sequences set forth in SEQ ID NOs: 1-10 (FIG. 4).
18. The monovalent antibody of item 17, comprising an amino acid sequence having at least 90% identity with one of the sequences set forth in SEQ ID NOs: 1-10 (FIG. 4).
19. The monovalent antibody of item 17, comprising one of the amino acid sequences set forth in SEQ ID NOs: 1-10 (FIG. 4).
20. The monovalent antibody according to any one of items 1 to 19, wherein said monovalent antibody further binds to GAL-16.
21. The monovalent antibody according to any one of items 1 to 20, wherein said monovalent antibody is fused to at least one antibody constant domain, or a fragment thereof.
22. The monovalent antibody according to item 21, wherein the at least one antibody constant domain or fragment thereof comprises a Fragment crystallizable (Fc) region, for example a Fc fragment comprising a CH₂ domain and CH₃ domain of a human antibody.
23. The monovalent antibody of any one of items 1 to 22, wherein said antibody is conjugated, for example to a label, a nanoparticle, a drug, a peptide, a nucleic acid, a toxin, an enzyme, a radioisotope, a half-life extending moiety, or a targeting agent, such as an E3 ubiquitin ligase binding moiety.
24. A nucleic acid comprising a nucleotide sequence encoding the monovalent antibody defined in any one of items 1 to 23.
25 The nucleic acid of item 24, which is in the form of mRNA.
26. The nucleic acid of item 24 or 25, which is encapsulated into lipid vesicles.
27. A vector comprising the nucleic acid of item 24.
28. A cell comprising the nucleic acid of any one of items 24 to 26 or the vector of item 27.
29. A pharmaceutical composition comprising the monovalent antibody defined in any one of items 1 to 23 or the nucleic acid of any one of items 24 to 26, and one or more pharmaceutically acceptable carriers, excipient, and/or diluents.
30. A method for binding human galectin-13 (GAL-13) and/or GAL-16 comprising contacting said GAL-13 and/or GAL-16 with the monovalent antibody of any one of items 1 to 23 or the composition of item 29.
31. A method for inhibiting galectin-13- and/or galectin-16-mediated apoptosis in a cell, said method comprising contacting said cell with an effective amount of the monovalent antibody of any one of items 1 to 23, the nucleic acid of any one of items 24 to 26, or the composition of item 29.
32. The method of item 31, wherein said cell is an immune cell.
33. The method of item 32, wherein said immune cell is a T lymphocyte.
34. A method for inhibiting the activity of human galectin-13 (GAL-13) and/or GAL-16 comprising contacting said GAL-13 and/or GAL-16 with an effective amount of the monovalent antibody of any one of items 1 to 23, the nucleic acid of any one of items 24 to 26, or the composition of item 29.
35. A method for treating a galectin-13 (GAL-13)- and/or galectin-16-expressing cancer in a subject, said method comprising administering to said subject an effective amount of the monovalent antibody of any one of items 1 to 23, the nucleic acid of any one of items 24 to 26, or the composition of item 29.
36. The method of item 35, wherein the GAL-13-expressing cancer is liver adenocarcinoma malignant melanoma, or a cancer of neural cells, and wherein said GAL-16-expressing cancer is chronic myeloid leukemia (CML), breast cancer, or B-cell lymphoma.
37. The method of item 35 or 36, wherein said monovalent antibody, nucleic acid or composition is administered in combination with a second anti-tumoral agent.
38. The method of any one of items 35 to 37, wherein the subject is a human subject.
39. The monovalent antibody of any one of items 1 to 23, or the composition of item 29, for use in binding human galectin-13 (GAL-13) and/or GAL-16.
40. The monovalent antibody of any one of items 1 to 23, or the composition of item 29, for use in inhibiting galectin-13- and/or galectin-16-mediated apoptosis in a cell.
41. The monovalent antibody or composition for use according to item 40, wherein said cell is an immune cell.
42. The monovalent antibody or composition for use according to item 41, wherein said immune cell is a T lymphocyte.
43. The monovalent antibody of any one of items 1 to 23, the nucleic acid of any one of items 24 to 26, or the composition of item 29, for use in inhibiting the activity of human galectin-13 (GAL-13) and/or GAL-16.
44. The monovalent antibody of any one of items 1 to 23, the nucleic acid of any one of items 24 to 26, or the composition of item 29, for use in the treatment of a galectin-13 (GAL-13)- or GAL-16-expressing cancer in a subject.
45. The monovalent antibody, nucleic acid or composition for use according to item 44, wherein the GAL-13-expressing cancer is liver adenocarcinoma malignant melanoma, or a cancer of neural cells, and wherein said GAL-16-expressing cancer is chronic myeloid leukemia (CML), breast cancer, or B-cell lymphoma.
46. The monovalent antibody, nucleic acid or composition for use according to item 44 or 45, wherein said monovalent antibody, nucleic acid or composition is administered in combination with a second anti-tumoral agent.
47. The monovalent antibody, nucleic acid or composition for use according to any one of items 44 to 46, wherein the subject is a human subject.
48. A method for detecting a human galectin-13- and/or galectin-16-expressing cell comprising contacting said cell with the monovalent antibody of any one of items 1 to 23.
49. The method of item 48, wherein said monovalent antibody is conjugated to a detectable label.
50. The method of item 49, wherein said detectable label is a fluorescent molecule or a radioisotope.
51. The method of any one of items 48 to 50, wherein said cell is a tumor cell.
52. The method of item 51, wherein said method is for diagnosing and/or monitoring the progression of a galectin-13- and/or galectin-16-positive cancer in a subject.
53. Use of the monovalent antibody of any one of items 1 to 14 for detecting a human galectin-13- and/or galectin-16-expressing cell.
54. The use of item 53, wherein said monovalent antibody is conjugated to a detectable label.
55. The use of item 54, wherein said detectable label is a fluorescent molecule or a radioisotope.
56. The use of any one of items 53 to 55, wherein said cell is a tumor cell.
57. The use of item 56, wherein said use is for diagnosing and/or monitoring the progression of a galectin-13-positive cancer in a subject.
58. A monovalent antibody that binds to human galectin-9 (GAL-9), wherein the monovalent antibody comprises one of the following combinations of complementarity determining regions (CDRs):
   a CDR1 comprising or consisting of an amino acid sequence having at least 70% identity with the sequence DTSGEYS (SEQ ID NO:56); a CDR2 comprising or consisting of an amino acid sequence having at least 70% identity with the sequence DQGGIEI (SEQ ID NO:57); and a CDR3 comprising or consisting of an amino acid sequence having at least 70% identity with the sequence HTFYDMPDGYRQ (SEQ ID NO:58);
   a CDR1 comprising or consisting of an amino acid sequence having at least 70% identity with the sequence STSADST (SEQ ID NO:59); a CDR2 comprising or consisting of an amino acid sequence having at least 70% identity with the sequence ENNGADR (SEQ ID NO:60); and a CDR3 comprising or consisting of an amino acid sequence having at least 70% identity with the sequence YWKIAYTKQYEFL (SEQ ID NO:61);
   a CDR1 comprising or consisting of an amino acid sequence having at least 70% identity with the sequence DGSGWDI (SEQ ID NO:62); a CDR2 comprising or consisting of an amino acid sequence having at least 70% identity with the sequence RHDTTYS (SEQ ID NO:63); and a CDR3 comprising or consisting of an amino acid sequence having at least 70% identity with the sequence TIPASWNKS (SEQ ID NO:64);
   a CDR1 comprising or consisting of an amino acid sequence having at least 70% identity with the sequence GTWRWTI (SEQ ID NO:65); a CDR2 comprising or consisting of an amino acid sequence having at least 70% identity with the sequence RTNSEYQ (SEQ ID NO:66); and a CDR3 comprising or consisting of an amino acid sequence having at least 70% identity with the sequence EPTAVWADKGQEMQS (SEQ ID NO:67).
59. The monovalent antibody of item 58, wherein said at least 70% identity is at least 90% identity.
60. The monovalent antibody of item 58 or 59, which comprises the following combinations of CDRs: a CDR1 comprising or consisting of the sequence DTSGEYS (SEQ ID NO:56); a CDR2 comprising or consisting of the sequence DQGGIEI (SEQ ID NO:57); and a CDR3 comprising or consisting of the sequence HTFYDMPDGYRQ (SEQ ID NO:58).
61. The monovalent antibody of item 58 or 59, which comprises the following combinations of CDRs: a CDR1 comprising or consisting of the sequence STSADST (SEQ ID NO:59); a CDR2 comprising or consisting of the sequence ENNGADR (SEQ ID NO:60); and a CDR3 comprising or consisting of the sequence YWKIAYTKQYEFL (SEQ ID NO:61).
62. The monovalent antibody of item 58 or 59, which comprises the following combinations of CDRs: a CDR1 comprising or consisting of the sequence DGSGWDI (SEQ ID NO:62); a CDR2 comprising or consisting of the sequence RHDTTYS (SEQ ID NO:63); and a CDR3 comprising or consisting of the sequence TIPASWNKS (SEQ ID NO:64).
63. The monovalent antibody of item 58 or 59, which comprises the following combinations of CDRs: a CDR1 comprising or consisting of the sequence GTWRWTI (SEQ ID NO:65); a CDR2 comprising or consisting of the sequence RTNSEYQ (SEQ ID NO:66); and a CDR3 comprising or consisting of the sequence EPTAVWADKGQEMQS (SEQ ID NO:67).
64. The monovalent antibody of any one of items 58 to 63, wherein the monovalent antibody is a single-domain antibody.
65. The monovalent antibody of any one of items 58 to 64, which comprises:
   (i) a framework region (FR) 1 comprising an amino acid sequence having at least 50% identity with the sequence MAEVQLQASGGGFVQPGGSLRLSCAASG (SEQ ID NO:52);
   (ii) a FR2 comprising an amino acid sequence having at least 50% identity with the sequence MGWFRQAPGKEREFVSAIS (SEQ ID NO:53);
   (iii) a FR3 comprising or consisting of an amino acid sequence having at least 50% identity with the sequence YYADSVKGRFTISRDNSKNTVYLQMNSLRAEDTATYYCA (SEQ ID NO: 54);
   (iv) a FR4 comprising an amino acid sequence having at least 50% identity with the sequence YWGQGTQVTVSS (SEQ ID NO:55); or
   (v) any combination of (i) to (iv).
66. The monovalent antibody of item 65, which comprises:
   (i) a FR1 comprising an amino acid sequence having at least 90% identity with the sequence MAEVQLQASGGGFVQPGGSLRLSCAASG (SEQ ID NO:52);
   (ii) a FR2 comprising an amino acid sequence having at least 90% identity with the sequence MGWFRQAPGKEREFVSAIS (SEQ ID NO:53);
   (iii) a FR3 comprising or consisting of an amino acid sequence having at least 90% identity with the sequence YYADSVKGRFTISRDNSKNTVYLQMNSLRAEDTATYYCA (SEQ ID NO: 54);
   (iv) a FR4 comprising an amino acid sequence having at least 90% identity with the sequence YWGQGTQVTVSS (SEQ ID NO:55); or
   (v) any combination of (i) to (iv).
67. The monovalent antibody of item 66, which comprises:
   (i) a FR1 comprising the sequence MAEVQLQASGGGFVQPGGSLRLSCAASG (SEQ ID NO: 52);
   (ii) a FR2 comprising the sequence MGWFRQAPGKEREFVSAIS (SEQ ID NO:53);
   (iii) a FR3 comprising the sequence YYADSVKGRFTISRDNSKNTVYLQMNSLRAEDTATYYCA (SEQ ID NO:54);
   (iv) a FR4 comprising the sequence YWGQGTQVTVSS (SEQ ID NO:55); or
   (v) any combination of (i) to (iv).
68. The monovalent antibody of any one of items 58 to 67, comprising an amino acid sequence having at least 80% identity with one of the sequences set forth in SEQ ID NOs: 12-15 (FIG. 16).
69. The monovalent antibody of item 68, comprising an amino acid sequence having at least 90% identity with one of the sequences set forth in SEQ ID NOs: 12-15 (FIG. 16).
70. The monovalent antibody of item 68, comprising one of the amino acid sequences set forth in SEQ ID NOs: 12-15 (FIG. 16).
71. The monovalent antibody according to any one of items 58 to 70, wherein said monovalent antibody is fused to at least one antibody constant domain, or a fragment thereof.
72. The monovalent antibody according to item 71, wherein the at least one antibody constant domain or fragment thereof comprises a Fragment crystallizable (Fc) region.
73. The monovalent antibody according to item 72, wherein the Fc fragment comprises a CH₂ domain and CH₃ domain of a human antibody.
74. The monovalent antibody of any one of items 58 to 73, wherein said antibody is conjugated to a label, a nanoparticle, a drug, a peptide, a nucleic acid, a toxin, an enzyme, a radioisotope, or a half-life extending moiety.
75. A nucleic acid comprising a nucleotide sequence encoding the monovalent antibody defined in any one of items 58 to 74.
76. The nucleic acid of item 76, which is in the form of mRNA.
77. The nucleic acid of item 75 or 76, which is encapsulated into lipid vesicles.
78. A vector comprising the nucleic acid of item 75.
79. A cell comprising the nucleic acid of any one of items 75 to 77 or the vector of item 78.
80. A pharmaceutical composition comprising the monovalent antibody defined in any one of items 58 to 74 or the nucleic acid of any one of items 75 to 77, and one or more pharmaceutically acceptable carriers, excipient, and/or diluents.
81. A method for binding human galectin-9 (GAL-9) comprising contacting said GAL-9 with the monovalent antibody of any one of items 58 to 74 or the composition of item 80.
82. A method for inhibiting galectin-9-mediated apoptosis in a cell, said method comprising contacting said cell with an effective amount of the monovalent antibody of any one of items 58 to 74, the nucleic acid of any one of items 75 to 77, or the composition of item 80.
83. The method of item 82, wherein said cell is an immune cell.
84 The method of item 83, wherein said immune cell is a T lymphocyte.
85. A method for inhibiting the activity of human galectin-9 (GAL-9) comprising contacting said GAL-9 with an effective amount of the monovalent antibody of any one of items 58 to 74, the nucleic acid of any one of items 75 to 77, or the composition of item 80.
86. A method for treating a galectin-9 (GAL-9)-expressing cancer in a subject, said method comprising administering to said subject an effective amount of the monovalent antibody of any one of items 58 to 74, the nucleic acid of any one of items 75 to 77, or the composition of item 80.
87. The method of item 86, wherein the GAL-9-expressing cancer is a hematological cancer.
88. The method of item 87, wherein the hematological cancer is chronic lymphocytic leukemia (CLL), myelodysplastic syndromes (MDS), Hodgkin lymphoma, Non-Hodgkin lymphoma, or acute myeloid leukemia (AML).
89. The method of item 86, wherein the GAL-9-expressing cancer is melanoma, lung cancer, breast cancer, colon cancer or liver cancer.
90. The method of any one of items 86 to 89, wherein said monovalent antibody, nucleic acid or composition is administered in combination with a second anti-tumoral agent.
91. A method for treating an inflammatory disease associated with GAL-9 activity in a subject, said method comprising administering an effective amount of the monovalent antibody of any one of items 58 to 74, the nucleic acid of any one of items 75 to 77, or the composition of item 80 to the subject.
92. The method of item 91, wherein the inflammatory disease is rheumatoid arthritis.
93. The method of any one of items 86 to 92, wherein the subject is a human subject.
94. The monovalent antibody of any one of items 58 to 74, or the composition of item 80, for use in binding human galectin-9 (GAL-9).
95. The monovalent antibody of any one of items 1 to 23, the nucleic acid of any one of items 75 to 77, or the composition of item 80, for use in inhibiting galectin-13-mediated apoptosis in a cell.
96. The monovalent antibody, nucleic acid or composition for use according to item 95, wherein said cell is an immune cell.
97. The monovalent antibody, nucleic acid or composition for use according to item 96, wherein said immune cell is a T lymphocyte.
98. The monovalent antibody of any one of items 58 to 74, the nucleic acid of any one of items 75 to 77, or the composition of item 80, for use in inhibiting the activity of human galectin-9 (GAL-9).
99. The monovalent antibody of any one of items 58 to 74, the nucleic acid of any one of items 75 to 77, or the composition of item 80, for use in the treatment of a galectin-9 (GAL-9)-expressing cancer in a subject.
100. The monovalent antibody, nucleic acid or composition for use according to item 99, wherein the GAL-9-expressing cancer is a hematological cancer.
101. The monovalent antibody, nucleic acid or composition for use according to item 100, wherein the hematological cancer is chronic lymphocytic leukemia (CLL), myelodysplastic syndromes (MDS), Hodgkin lymphoma, Non-Hodgkin lymphoma, or acute myeloid leukemia (AML).
102. The monovalent antibody, nucleic acid or composition for use according to item 99, wherein the GAL-9-expressing cancer is melanoma, lung cancer, breast cancer, colon cancer or liver cancer.
103. The monovalent antibody, nucleic acid or composition for use according to any one of items 99 to 102, wherein said monovalent antibody, nucleic acid or composition is administered in combination with a second anti-tumoral agent.
104. The monovalent antibody of any one of items 58 to 74, the nucleic acid of any one of items 75 to 77, or the composition of item 80, for use in the treatment of an inflammatory disease associated with GAL-9 activity.
105. The monovalent antibody, nucleic acid or composition for use according to item 104, wherein the inflammatory disease is rheumatoid arthritis.
106. The monovalent antibody, nucleic acid or composition for use according to any one of items 99 to 105, wherein the subject is a human subject.
107. A method for detecting a human galectin-13-expressing cell comprising contacting said cell with the monovalent antibody of any one of items 58 to 74.
108. The method of item 107, wherein said monovalent antibody is conjugated to a detectable label.
109. The method of item 108, wherein said detectable label is a fluorescent molecule or a radioisotope.
110. The method of any one of items 107 to 109, wherein said cell is a tumor cell.
111. The method of item 110, wherein said method is for diagnosing and/or monitoring the progression of a galectin-9-positive cancer in a subject.
112. Use of the monovalent antibody of any one of items 58 to 74 for detecting a human galectin-9-expressing cell.
113. The use of item 112, wherein said monovalent antibody is conjugated to a detectable label.
114. The use of item 113, wherein said detectable label is a fluorescent molecule or a radioisotope.
115. The use of any one of items 112 to 114, wherein said cell is a tumor cell.
116. The use of item 115, wherein said use is for diagnosing and/or monitoring the progression of a galectin-9-positive cancer in a subject.
117. A method for treating a disease or condition associated with pathological neovascularization or angiogenesis in a subject comprising administering to said subject an effective amount of the monovalent antibody of any one of items 58 to 74, the nucleic acid of any one of items 75 to 77, or the composition of item 80.
118. The monovalent antibody of any one of items 58 to 74, the nucleic acid of any one of items 75 to 77, or the composition of item 80, for the manufacture of a medicament for treating a disease or condition associated with pathological neovascularization or angiogenesis.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows a Western blot using a Streptavidin-HRP conjugate (Thermo Fischer) showing successful biotinylation of human Galectin-13 (GAL-13) that was used for selection of galectins-specific sdAbs by phage display. Lane 1:212 ng of Galectin13-Biotin without beads; Lane 2:212 ng of Galectin13-Biotin on beads for round 1; Lane 3:46 ng of Galectin13-Biotin on beads for rounds 2 and 3; Lane 4:193 ng of MBP-HA-Flag-Biotin without beads; Lane 5:193 ng of GST-His-Flag-MBP-Biotin on beads for all three rounds.

FIGS. 2A-B show the enrichment during three consecutive rounds of Phage Display selection by the ratio Output/Input. The selection was carried out using a phage display library containing 3×10⁹ camelid VHH sequences. To control the enrichment of specific anti-Galectin-13 VHHs during each round of Phage Display, an output/input ratio was measured (FIG. 2A) and an ELISA on the pool of each round was performed (FIG. 2B). The third round of Phage Display was performed twice, and at the end of the screening, a total of 136 E. coli clones were picked randomly and analyzed by non-adsorbed phage ELISA.

FIG. 3 shows the binding to Galectin-13 of the 10 best positive VHH clones by a non-adsorbed phage ELISA. First, the binding to GAL-13 of the 136 selected VHH clones was tested using Hybrigenics' non-adsorbed phage ELISA that allows for the proper folding of the native GAL-13 protein. The binding of the positive VHHs was reconfirmed in a second non-adsorbed phage ELISA and 18 VHH were positive in the phage ELISA. They correspond to 17 different VHH. The binding of the positive VHHs was reconfirmed in a second non-adsorbed phage ELISA as shown.

FIG. 4 shows the amino acid sequences of the G13N1 (SEQ ID NO:1), G13N2 (SEQ ID NO: 2), G13N3 (SEQ ID NO:3), G13N4 (SEQ ID NO:4), G13N5 (SEQ ID NO:5), G13N6 (SEQ ID NO: 6), G13N7 (SEQ ID NO:7), G13N8 (SEQ ID NO:8), G13N9 (SEQ ID NO:9) and G13N10 (SEQ ID NO: 10) sdAbs that bind to GAL-13. Reference (SEQ ID NO: 11) is a sdAb with the CDR region replaced by “X”. The residues forming the CDRs and FRs have been assigned according to the nomenclature set forth in Moutel et al., eLife 2016; 5: e162.

FIG. 5 shows examples of GAL-13-specific single-domain antibodies (sdAbs) purification. DNA sequences encoding GAL-13-specific sdAbs were inserted into the basic pHEN2 vector for production in E. coli. The SDS-PAGE analysis shows purification of GAL-13-specific Nb #4 (G13N4) (A), Nb #5 (G13N5) (B), Nb #7 (G13N7) (C) and Nb #10 (G13N10) (D) by standard metal (Ni)-affinity chromatography using imidazole gradient (40 to 500 mM).

FIG. 6 shows that recombinant human GAL-13 induces apoptosis of human T cells. 250,000 Jurkat cells were exposed overnight to increasing doses of GAL-13 (1, 3, 5, 10 and 20 μM) compared to vehicle control. Live cells were co-labeled with Annexin V and PI staining, and analyzed using BD FACSalibur™ flow cytometry. All the experiments were performed in four biological replicates with duplicates.

FIG. 7 shows the ability of Gal-13 sdAbs to inhibit GAL-13-induced apoptosis of human T cells. 250,000 Jurkat cells were exposed overnight with [3 μM] of GAL-13 or co-treated with [3 μM] of GAL-13 and [30 μM] of each sdAb (G13N1 to G13N10). GAL-13 and sdAbs were preincubated together 1 h at 37° C. before cell treatment. Live cells were co-labeled with Annexin V and PI staining, and analyzed using BD FACSalibur™ flow cytometry. All the experiments were performed in three biological replicates with duplicates. All data are expressed as mean±S.E.M with p<0.05. Each experiment was performed in triplicate.

FIG. 8 shows the binding of GAL-13 sdAbs to human galectins as measured by ELISA. Briefly, human galectins were immobilized at 0.1 μM for 16 h at 4° C. After a blocking step with PBS containing 10% (v/v) BSA-0.05% Tween™ 20 (blocking buffer), 1 μM of GAL-13 sdAbs were added to each well and incubated for 1 h at room temperature. The binding of GAL-13 sdAbs was revealed using a goat anti-His-tag polyclonal (1/1000) antibody and a donkey anti-goat IgG (1/5000) conjugated to horseradish peroxidase (HRP). The colorimetric assay was carried out with 3,3′,5,5′-tetramethylbenzidine (TMB) according to the manufacturer's recommendations. This data is representative of three independent experiments.

FIG. 9 shows the binding of human GAL-13 on Asialofetuin (ASF). ASF was immobilized at [10 μg/ml] for 16 h at 4° C. as measured by ELISA. Asialofetuin is a glycoprotein with three asparagine-linked triantennary complex carbohydrate chains and terminal N-acetylgalactosamine residues (LacNac), conferring it with the ability to bind multiple galectins. After a blocking step with PBS containing 10% (v/v) BSA-0.05% Tween™ 20 (blocking buffer) for 2 h at room temperature, an increasing concentration of GAL-13 was added and incubated for 1 h at room temperature. Binding of GAL-13 was revealed using a mouse anti-Galectin-13 monoclonal (1/1000) antibody and an anti-mouse IgG (1/10000) conjugated to HRP. The colorimetric assay was carried out with 3,3′,5,5′-tetramethylbenzidine according to the manufacturer's recommendations. Experiments were performed in triplicate.

FIG. 10 shows the capacity of GAL-13 sdAbs to prevent the binding of GAL-13 on ASF as measured by ELISA. Briefly, ASF was immobilized at [10 μg/ml] for 16 h at 4° C. During the blocking step with PBS containing 10% (v/v) BSA-0.05% Tween™ 20 (blocking buffer), Gal-13 at [0.3 μM] was mixed with each sdAb at [10 UM] or [20 μM] 1 h at 4° C. Binding of GAL-13 was revealed using a mouse anti-Galectin-13 monoclonal (1/1000) antibody and an anti-mouse IgG (1/10000) conjugated to HRP. The colorimetric assay was carried out with 3,3′,5,5′-tetramethylbenzidine according to the manufacturer's recommendations. Data shown are representative of three independent experiments.

FIG. 11 demonstrates the ability of sdAbs to inhibit GAL-13 binding to ASF synergistically. Before adding the mix GAL-13/sdAbs on ASF previously coated at [10 μg/ml], GAL-13 [1 μM] was pre-incubated with mixed sdAbs at [10 UM] 1 h et 4° C. Binding of GAL-13 was revealed using a mouse anti-Galectin-13 monoclonal (1/1000) antibody and an anti-mouse IgG (1/10000) conjugated to HRP. The colorimetric assay was carried out with 3,3′,5,5′-tetramethylbenzidine according to the manufacturer's recommendations. Experiments were performed in triplicate.

FIGS. 12A-D the binding of GAL-13-FITC on HepG2 cancer cell line and the ability of sdAbs to prevent its binding. Human recombinant GAL-13 was labeled with FITC by successive dialysis. HepG2 cells were incubated with increasing doses of GAL-13-FITC (FIG. 12A) and an incubation during 1 h at 4° C. with unlabeled GAL-13 and GAL-1 demonstrates a GAL-13 specific binding (FIG. 12B). Pre-incubation of GAL-13-FITC with increasing doses of G13N4 (FIG. 12C) and G13N7 (FIG. 12D) demonstrates a 50% decrease of GAL-13 FITC binding on HepG2 cells.

FIG. 13 shows a Western blot using a Streptavidin-HRP conjugate (Thermo Fischer) showing successful biotinylation of human galectin-9 (GAL-9) with a Histidine tag that was used for the selection of galectin-9-specific sdAbs by phage display. Lane 1: Biotinylated His-Galectin-9 without Streptavidin Magnetic Beads (Dynabeads® M-280 Streptavidin, Life Technologies); Lane 2: Biotinylated His-Galectin-9 on beads for round 1; Lane 3: Biotinylated His-Galectin-9 on beads for rounds 2 and 3; Lane 4: His-MBP-GST-Flag-Biotin without beads; Lane 5: His-MBP-GST-Flag-Biotin on beads that was used for in an initial round of Phage Display to deplete the library from unspecific binders. Expected molecular weights of proteins: His-Galectin 9:38 kDa (60 kDa reducing conditions); His-MBP-GST-Flag: 69 kDa.

FIG. 14 shows the enrichment during three consecutive rounds of Phage Display selection by the ratio Output/Input. The selection was carried out using a phage display library containing 3×10⁹ camelid VHH sequences. For selection, the phage library was first incubated with the His-MBP-GST-Flag-Biotin beads to remove unspecific binders. Unbound VHHs were then incubated with the His-Galectin-9-Biotin beads. A total of three rounds of Phage Display were performed. The depletion step was repeated before each Phage Display round to remove non-specific VHHs.

FIG. 15 shows the binding of the 4 positive clones that were identified following the testing of 90 clones in a 384-well plate ELISA with an HRP-conjugated anti-M13 antibody (GE Healthcare) and a colorimetric substrate (TMB, TetraMethylBenzidine, Thermo Fischer). All four clones showed significant binding in the presence of His-Galectin-9-Biotin and a very low signal in the presence of His-MBP-GST-Flag-Biotin as measured by ELISA.

FIG. 16 shows the amino acid sequence of the G9N1 (SEQ ID NO: 12), G9N2 (SEQ ID NO: 13), G9N3 (SEQ ID NO: 14) and G9N4 (SEQ ID NO: 15) sdAbs that bind to Gal-9. Reference (SEQ ID NO:16) is a sdAb with the CDR region replaced by “X”. The residues forming the CDRs and FRs have been assigned according to the nomenclature outlined in Moutel et al., eLife 2016; 5: e162.

FIG. 17 shows the production and purification of GAL-9-specific sdAbs. DNA sequences encoding GAL-9-specific sdAbs were inserted into the basic pHEN2 vector for production in E. coli. The SDS-PAGE analysis shows purified GAL-9-specific G9N1, G9N2, G9N3 and G9N4 by standard metal (Ni)-affinity chromatography.

FIG. 18 shows the binding of human GAL-9 to sdAbs as measured by ELISA. Briefly, Nbs were immobilized at 1 μM for 1 h at RT, followed by a blocking step with PBS containing 10% (v/v) BSA and 0.05% (v/v) Tween™ 20. Increasing concentrations of GAL-9 were added to each well and incubated for 2 h at RT. The binding of GAL-9 was revealed using a rabbit anti-human GAL-9 (1/500) antibody and a goat anti-rabbit IgG (1/5000) antibody conjugated to HRP. The colorimetric assay was carried out with 3,3′,5,5′-tetramethylbenzidine per the manufacturer's recommendations. Data (mean values±SD) from one experiment is shown. Statistical differences were assessed using Tukey's multiple comparisons test. RT=room temperature.

FIGS. 19A-B show the binding of recombinant N-terminal domain of human GAL-9 (GAL-9N, FIG. 19A) and recombinant C-terminal domain of human GAL-9 (GAL-9C, FIG. 19B) to GAL-9-specific sdAbs as measured by ELISA. Briefly, sdAbs were immobilized at 1 μM for 1 h at RT, followed by blocking with PBS containing 10% (v/v) BSA and 0.05% (v/v) Tween™ 20. 5 μM of recombinant GAL-9N or GAL-9C were incubated for 1 h at RT. The binding of recombinant Gal-9N was revealed using a rabbit anti-human GAL-9 (1/500) antibody and an HRP-conjugated goat anti-rabbit IgG (1/5000) antibody. The binding of recombinant GAL-9C was revealed using a mouse anti-human GAL-9C (1/200) antibody and an HRP-conjugated goat anti-mouse IgG (1/2000) antibody. The colorimetric assay was carried out with 3,3′,5,5′-tetramethylbenzidine per the manufacturer's recommendations. Data (mean values±SD) from three independent experiments is shown. Statistical differences were assessed using Tukey's multiple comparisons test. RT=room temperature.

FIG. 20 shows the binding of human galectins to GAL-9-specific sdAbs as measured by ELISA. Briefly, Nbs were immobilized at 1 μM for 1 h at RT, followed by blocking with PBS containing 10% (v/v) BSA and 0.05% (v/v) Tween™ 20. 5 μM of galectin were incubated for 1 h at RT. Binding of galectins was revealed using their respective primary and secondary antibodies. The colorimetric assay was carried out with 3,3′,5,5′-tetramethylbenzidine per the manufacturer's recommendations. Data (mean values±SD) from one experiment is shown. Statistical differences were assessed using Dunnett's multiple comparisons test. RT=room temperature

FIG. 21 shows an amino acid sequence alignment of human galectin-1 (GAL-1), galectin-7 (GAL-7), galectin-9 (GAL-9), galectin-13 (GAL-13) and galectin-16 (GAL-16).

### DETAILED DISCLOSURE

The use of the terms “a” and “an” and “the” and similar referents in the context of describing the invention are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein, a general chemical structure with various substituents and various radicals enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

All methods described herein can be performed in any suitable order unless otherwise indicated herein or otherwise clearly contradicted by context.

The use of any and all examples, or exemplary language (“e.g.”, “such as”, etc.) provided herein, is intended merely to better illustrate the invention and does not pose a limitation on the scope of the invention unless otherwise claimed.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Unless otherwise defined, all technical and scientific terms used herein have the same meaning as commonly understood by one of ordinary skill in the art to which this invention belongs.

Any and all combinations and subcombinations of the embodiments and features disclosed herein are encompassed by the present disclosure.

In the studies described herein, the present inventors have developed cameloid antibodies (also called single domain antibodies (sdAbs), VHH, or Nanobodies (NAbs), which are particularly well-suited for dimer-interference and/or blocking the GBS of galectins given their small size. First, sdAbs bind antigenic epitopes by virtue of a single (monovalent) and variable domain encoded in the heavy chain fragment [Hamers-Casterman et al., 2013]. This is an important issue in the case of galectin because, in contrast to conventional (multivalent) antibodies (Abs), binding of monovalent sdAbs on galectin-bound cell surface glycoreceptors cannot trigger intracellular signals induced by cross-linking of glycoreceptors. Secondly, sdAbs can recognize epitopes that may be inaccessible to conventional Abs because of their extended convex-shaped paratope. This is possible because the hypervariable region of sdAbs is made of a single stretch of amino acids (a.a.) composed of flexible peptide loops, including a relatively long complementary determining region (CDR)-3 loop that is extended and made of 17 a.a on average (compared to 12 a.a. in humans). This confers sdAbs with a unique antigen-binding mode capable of targeting hidden (poorly immunogenic) epitopes. The structure of their antigen-binding region is thus ideally suited for targeting epitopes that are not accessible by conventional antibodies, which harbor relatively flat paratopes. Moreover, in contrast to conventional (multivalent) antibodies, binding of monovalent sdAbs on galectin-bound cell surface glycoreceptors cannot trigger intracellular signals induced by cross-linking of glycoreceptors. Finally, another important consideration is that sdAbs usually exhibit high affinity for their ligands, often in the subnanomolar range. All of these features suggest that sdAbs are ideally suited for inhibiting galectins such as GAL-9 and GAL-13. In the studies disclosed in the present disclosure, the generation of specific and high-affinity camelid antibodies against human GAL-9 and GAL-13 is reported. These sdAbs were shown to be specific for their target galectins, and to interfere with their activity. Certain antibodies raised against GAL-13 were also shown to have the ability to bind to GAL-16, which has a sequence identity with GAL-13.

Accordingly, in a first aspect, the present disclosure provides a monovalent antibody that specifically binds to human GAL-9 or human GAL-13. In an embodiment, the present disclosure provides a monovalent antibody that binds to the glycan-binding site (GBS) of human GAL-9 or GAL-13. In another embodiment, the monovalent antibody inhibits or interferes with human GAL-9 or human GAL-13 activity, for example, GAL-13-induced killing of cells such as human activated T cells. In another embodiment, the monovalent antibody inhibits or interferes with the binding of human GAL-9 or human GAL-13 to one of its ligands.

In another aspect, the present disclosure provides a monovalent antibody that specifically binds to human GAL-13 and GAL-16. In an embodiment, the present disclosure provides a monovalent antibody that specifically binds to the glycan-binding site (GBS) of human GAL-13 and GAL-16. In another embodiment, the monovalent antibody inhibits or interferes with human GAL-13 and GAL-16 activity, for example, GAL-13- and GAL-16-induced killing of cells such as human activated T cells. In another embodiment, the monovalent antibody inhibits or interferes with human GAL-13 and GAL-16 binding to one of their ligands.

The term “monovalent antibody” refers to an antibody that comprises a single monomeric variable antibody domain and, thus, a single set of complementary determining regions (CDRs) that binds to the target antigen (human GAL-9 or human GAL-13). Examples of monovalent antibodies include single-domain antibodies (sdAbs, also called nanobodies), camelid antibodies (e.g., from dromedaries, camels, llamas, alpacas), V_{H}H fragments and V_{NAR} fragments. In an embodiment, the monovalent antibody is a sdAb. Single domain antibodies may be derived from any species, including mice, humans, camels, llamas, goats, rabbits, and bovines. For example, naturally occurring V_{H}H molecules can be derived from antibodies raised in Camelidae species, such as in camel, dromedary, alpaca, and guanaco. Synthetic VHH molecules may also be identified/generated using a library of humanized sdAbs (see, e.g., Moutel et al., eLife 2016; 5: e16228; Salema et al., MAbs vol. 8, No. 7, 1286-1301; Gene, R W et al., (2015) J Immunol Methods 416, 29-39; Kumaran, J. (2012). Methods Mol Biol 911, 105-124). The monovalent antibody of the present disclosure may be fused or combined with another antibody or antibody fragment that binds to another antigen (e.g., in a bispecific antibody). For example, the monovalent antibody of the present disclosure may be fused or combined with a sdAb recognizing another antigen such as an effector of the immune response (e.g., CD3).

GAL-9 is a member of the galectin family of animal lectins with conserved carbohydrate-recognition domains (CRDs) for β-galactosides. Structurally, GAL-9 consists of two CRDs connected by a linker sequence and is able to crosslink glycoproteins to form multivalent galectin-glycoprotein lattices that regulate multiple cellular processes. GAL-9 is highly expressed in rheumatoid arthritis (RA) synovial fluid (SF) and synovial tissue (ST) compared with normal or osteoarthritic ST (Seki M, et al., Arthritis Rheum. 2007; 56 (12): 3968-76), suggesting a role for GAL-9 in RA. GAL-9 has been shown to mediate angiogenesis, increase monocyte migration in vitro, and induce acute inflammatory arthritis in mice, suggesting a role for GAL-9 in angiogenesis, joint inflammation, and possibly other inflammatory diseases (O'Brien et al., Arthritis Research & Therapy volume 20, Article number: 31 (2018).

The expression of GAL-9 has been detected on various hematological malignancies, such as Chronic lymphocytic leukemia (CLL), myelodysplastic syndromes (MDS), Hodgkin and Non-Hodgkin lymphomas, acute myeloid leukemia (AML) or solid tumors, such as melanoma, lung cancer, breast cancer, colon cancer and hepatocellular carcinoma, and high GAL-9 expression has been associated with poor prognosis in multiple human cancers. Interaction of GAL-9 with the T-cell immunoglobulin and mucin-domain containing-3 (TIM-3) receptor (HAVCR2) has been shown to attenuate T-cell expansion and effectors function in tumor microenvironment and chronic infections. Moreover, GAL-9 may contribute to tumorigenesis by tumor cell transformation, cell-cycle regulation, angiogenesis, and cell adhesion.

Gal-13, originally described as placental protein 13 (PP13), is a prototypic galectin that dimerizes via the formation of two intermolecular disulfide bonds formed by Cys136 and Cys138. It is strongly expressed in the syncytiotrophoblast at the lining of the maternal-fetal interface. Gal-13 has been shown to induce the apoptosis of pre-activated T lymphocytes and to increase IL-1α and IL-6 secretion from peripheral blood mononuclear cells (PBMCs) in pregnant women. Gal-13 is also expressed in the spleen, kidney, and bladder, as well as in liver adenocarcinoma, neurogenic tumors, and malignant melanoma.

GAL-16 is expressed in the placenta as well as in brain tissues and the retina. Like other galectins such as GAL-13, GAL-16 can induce apoptosis of CD3⁺ T cells. Alterations in the expression or mutations of GAL-16 have been detected in diseases and conditions such as Alzheimer's Disease (AD), psychiatric disorders, chronic myeloid leukemia (CML), breast cancer, large B-cell lymphoma and type 2 diabetes (Kaminker J D and Timoshenko A V. Biomolecules. 2021; 11 (12): 1909).

In an embodiment, the monovalent antibody disclosed herein may be used to treat any of the diseases/cancers defined above.

In an embodiment, the monovalent antibody binds to GAL-13 and comprises one of the following combinations of complementarity-determining regions (CDRs):
a CDR1 comprising or consisting of an amino acid sequence having at least 70%, 75%, 80%, 85% or 90% identity with the sequence FGSNRST (SEQ ID NO:22); a CDR2 comprising or consisting of an amino acid sequence having at least 70%, 75%, 80%, 85% or 90% identity with the sequence SLSSDPP (SEQ ID NO:23); and a CDR3 comprising or consisting of an amino acid sequence having at least 80%, 85% or 90% identity with the sequence PAGMRGVME (SEQ ID NO: 24);
a CDR1 comprising or consisting of an amino acid sequence having at least 70%, 75%, 80%, 85% or 90% identity with the sequence STSRSSG (SEQ ID NO:25); a CDR2 comprising or consisting of an amino acid sequence having at least 70%, 75%, 80%, 85% or 90% identity with the sequence, YRGSGAR (SEQ ID NO:26); and a CDR3 comprising or consisting of an amino acid sequence having at least 80%, 85% or 90% identity with the sequence MWQRQIQPGTRPVMK (SEQ ID NO:27);
a CDR1 comprising or consisting of an amino acid sequence having at least 70%, 75%, 80%, 85% or 90% identity with the sequence RGSRWYG (SEQ ID NO:28); a CDR2 comprising or consisting of an amino acid sequence having at least 70%, 75%, 80%, 85% or 90% identity with the sequence AQHSTRA (SEQ ID NO:29); and a CDR3 comprising or consisting of an amino acid sequence having at least 80%, 85% or 90% identity with the sequence GYMSKMGERKWK (SEQ ID NO:30);
a CDR1 comprising or consisting of an amino acid sequence having at least 70%, 75%, 80%, 85% or 90% identity with the sequence SSYAGSA (SEQ ID NO:31); a CDR2 comprising or consisting of an amino acid sequence having at least 70%, 75%, 80%, 85% or 90% identity with the sequence SDPDTKA (SEQ ID NO:32); and a CDR3 comprising or consisting of an amino acid sequence having at least 80%, 85% or 90% identity with the sequence ISGQYPYAR (SEQ ID NO: 33);
a CDR1 comprising or consisting of an amino acid sequence having at least 70%, 75%, 80%, 85% or 90% identity with the sequence YTSNEDS (SEQ ID NO:34); a CDR2 comprising or consisting of an amino acid sequence having at least 70%, 75%, 80%, 85% or 90% identity with the sequence RGPSFRT (SEQ ID NO:35); and a CDR3 comprising or consisting of an amino acid sequence having at least 80%, 85% or 90% identity with the sequence IRLGRLYRYGDLTPKSAY (SEQ ID NO:36);
a CDR1 comprising or consisting of an amino acid sequence having at least 70%, 75%, 80%, 85% or 90% identity with the sequence RTSSLTS (SEQ ID NO:37); a CDR2 comprising or consisting of an amino acid sequence having at least 70%, 75%, 80%, 85% or 90% identity with the sequence FSGSMFT (SEQ ID NO:38); and a CDR3 comprising or consisting of an amino acid sequence having at least 80%, 85% or 90% identity with the sequence DKWMHKPAN (SEQ ID NO: 39);
a CDR1 comprising or consisting of an amino acid sequence having at least 70%, 75%, 80%, 85% or 90% identity with the sequence RTWNSYT (SEQ ID NO:40); a CDR2 comprising or consisting of an amino acid sequence having at least 70%, 75%, 80%, 85% or 90% identity with the sequence GEPGGIR (SEQ ID NO:41); and a CDR3 comprising or consisting of an amino acid sequence having at least 80%, 85% or 90% identity with the sequence DRWPNKHDR (SEQ ID NO: 42);
a CDR1 comprising or consisting of an amino acid sequence having at least 70%, 75%, 80%, 85% or 90% identity with the sequence TTWKQET (SEQ ID NO:43); a CDR2 comprising or consisting of an amino acid sequence having at least 70%, 75%, 80%, 85% or 90% identity with the sequence RGPNYYP (SEQ ID NO:44); and a CDR3 comprising or consisting of an amino acid sequence having at least 80%, 85% or 90% identity with the sequence DEWMGSINARHGSGY (SEQ ID NO:45);
a CDR1 comprising or consisting of an amino acid sequence having at least 70%, 75%, 80%, 85% or 90% identity with the sequence RGSALTT (SEQ ID NO:46); a CDR2 comprising or consisting of an amino acid sequence having at least 70%, 75%, 80%, 85% or 90% identity with the sequence GDTGLYV (SEQ ID NO:47); and a CDR3 comprising or consisting of an amino acid sequence having at least 80%, 85% or 90% identity with the sequence GKWLGGRQADTR (SEQ ID NO: 48); or
a CDR1 comprising or consisting of an amino acid sequence having at least 70%, 75%, 80%, 85% or 90% identity with the sequence RTSNQT (SEQ ID NO:49); a CDR2 comprising or consisting of an amino acid sequence having at least 70%, 75%, 80%, 85% or 90% identity with the sequence ARFDEYG (SEQ ID NO:50); and a CDR3 comprising or consisting of an amino acid sequence having at least 80%, 85% or 90% identity with the sequence LGWPQKSTWRAYTAE (SEQ ID NO:51).

In another embodiment, the monovalent antibody binds to GAL-9 and comprises one of the following combinations of CDRs:
a CDR1 comprising or consisting of an amino acid sequence having at least 70%, 75%, 80%, 85% or 90% identity with the sequence DTSGEYS (SEQ ID NO:56); a CDR2 comprising or consisting of an amino acid sequence having at least 70%, 75%, 80%, 85% or 90% identity with the sequence DQGGIEI (SEQ ID NO:57); and a CDR3 comprising or consisting of an amino acid sequence having at least 80%, 85% or 90% identity with the sequence HTFYDMPDGYRQ (SEQ ID NO: 58);
a CDR1 comprising or consisting of an amino acid sequence having at least 70%, 75%, 80%, 85% or 90% identity with the sequence STSADST (SEQ ID NO:59); a CDR2 comprising or consisting of an amino acid sequence having at least 70%, 75%, 80%, 85% or 90% identity with the sequence ENNGADR (SEQ ID NO:60); and a CDR3 comprising or consisting of an amino acid sequence having at least 80%, 85% or 90% identity with the sequence YWKIAYTKQYEFL (SEQ ID NO:61);
a CDR1 comprising or consisting of an amino acid sequence having at least 70%, 75%, 80%, 85% or 90% identity with the sequence DGSGWDI (SEQ ID NO:62); a CDR2 comprising or consisting of an amino acid sequence having at least 70%, 75%, 80%, 85% or 90% identity with the sequence RHDTTYS (SEQ ID NO:63); and a CDR3 comprising or consisting of an amino acid sequence having at least 80%, 85% or 90% identity with the sequence TIPASWNKS (SEQ ID NO: 64); or
a CDR1 comprising or consisting of an amino acid sequence having at least 70%, 75%, 80%, 85% or 90% identity with the sequence GTWRWTI (SEQ ID NO:65); a CDR2 comprising or consisting of an amino acid sequence having at least 70%, 75%, 80%, 85% or 90% identity with the sequence RTNSEYQ (SEQ ID NO:66); and a CDR3 comprising or consisting of an amino acid sequence having at least 80%, 85% or 90% identity with the sequence EPTAVWADKGQEMQS (SEQ ID NO:67).

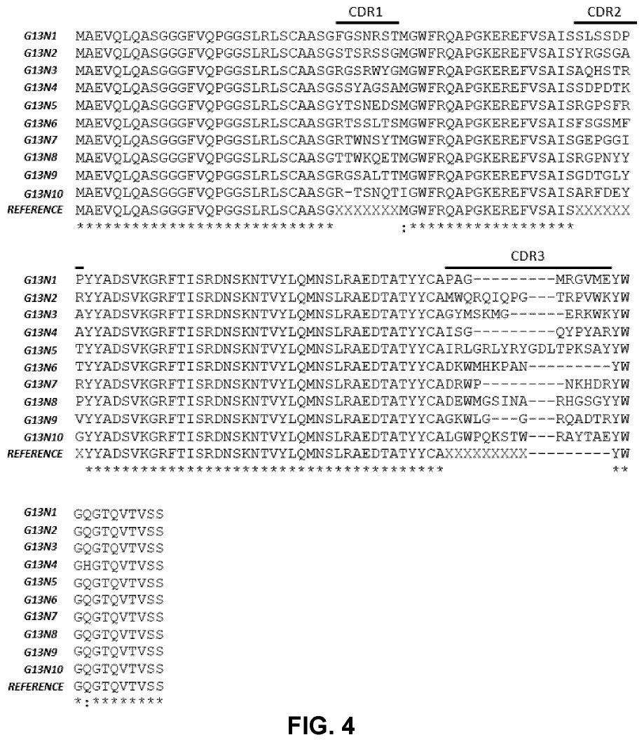

Although it will be appreciated that the skilled person will be able to provide for various single-domain antibodies based on the various CDR1, CDR2, CDR3 as disclosed herein, as well as the other sequences provided (including the various framework sequences and the full-length sequence of the single-domain antibodies), preferably the single-domain antibody has a CDR1, CDR2 and a CDR3 as shown in SEQ ID NOs: 1-10 (FIG. 4) or SEQ ID NOs: 12-15 (FIG. 16), and conservative sequence variants thereof. In other words, a single-domain antibody according to the present disclosure preferably comprises the CDR1 and the CDR2 and the CDR3 shown in FIG. 4 or FIG. 16. As will be appreciated by the skilled person, also included are conservative sequence variants of the CDR1, CDR2 and CDR3 combinations as disclosed in SEQ ID NOs: 1-10 (FIG. 4) or SEQ ID NOs: 12-15 (FIG. 16). These amino acid changes can typically be made without altering the biological activity, function, or other desired property of the antibody, such as its affinity or its specificity for antigen. In general, single amino acid substitutions in nonessential regions of an antibody do not substantially alter biological activity. Furthermore, substitutions of amino acids that are similar in structure or function are less likely to disrupt the antibody's biological activity. One or more of the CDRs may be mutated to increase the affinity and/or specificity of the monovalent antibody for GAL-13 or GAL-9, e.g., to generate an affinity-matured monovalent antibody.

In an embodiment, one or two residues in the above-noted CDRs sequences are substituted. In a further embodiment, one residue in the above-noted CDRs sequences is substituted.

In an embodiment, the monovalent antibody binds to GAL-13 and comprises one of the following combinations of CDRs:
a CDR1 comprising or consisting of the sequence FGSNRST (SEQ ID NO:22); a CDR2 comprising or consisting of the sequence SLSSDPP (SEQ ID NO:23); and a CDR3 comprising or consisting of the sequence PAGMRGVME (SEQ ID NO:24);
a CDR1 comprising or consisting of the sequence STSRSSG (SEQ ID NO:25); a CDR2 comprising or consisting of the sequence, YRGSGAR (SEQ ID NO:26); and a CDR3 comprising or consisting of the sequence MWQRQIQPGTRPVMK (SEQ ID NO:27);
a CDR1 comprising or consisting of the sequence RGSRWYG (SEQ ID NO:28); a CDR2 comprising or consisting of the sequence AQHSTRA (SEQ ID NO:29); and a CDR3 comprising or consisting of the sequence GYMSKMGERKWK (SEQ ID NO:30);
a CDR1 comprising or consisting of the sequence SSYAGSA (SEQ ID NO:31); a CDR2 comprising or consisting of the sequence SDPDTKA (SEQ ID NO:32); and a CDR3 comprising or consisting of the sequence ISGQYPYAR (SEQ ID NO:33);
a CDR1 comprising or consisting of the sequence YTSNEDS (SEQ ID NO:34); a CDR2 comprising or consisting of the sequence RGPSFRT (SEQ ID NO:35); and a CDR3 comprising or consisting of the sequence IRLGRLYRYGDLTPKSAY (SEQ ID NO:36);
a CDR1 comprising or consisting of the sequence RTSSLTS (SEQ ID NO:37); a CDR2 comprising or consisting of the sequence FSGSMFT (SEQ ID NO:38); and a CDR3 comprising or consisting of the sequence DKWMHKPAN (SEQ ID NO:39);
a CDR1 comprising or consisting of the sequence RTWNSYT (SEQ ID NO:40); a CDR2 comprising or consisting of the sequence GEPGGIR (SEQ ID NO:41); and a CDR3 comprising or consisting of the sequence DRWPNKHDR (SEQ ID NO:42);
a CDR1 comprising or consisting of the sequence TTWKQET (SEQ ID NO:43); a CDR2 comprising or consisting of the sequence RGPNYYP (SEQ ID NO:44); and a CDR3 comprising or consisting of the sequence DEWMGSINARHGSGY (SEQ ID NO:45);
a CDR1 comprising or consisting of the sequence RGSALTT (SEQ ID NO:46); a CDR2 comprising or consisting of the sequence GDTGLYV (SEQ ID NO:47); and a CDR3 comprising or consisting of the sequence GKWLGGRQADTR (SEQ ID NO:48); or
a CDR1 comprising or consisting of the sequence RTSNQT (SEQ ID NO:49); a CDR2 comprising or consisting of the sequence ARFDEYG (SEQ ID NO:50); and a CDR3 comprising or consisting of the sequence LGWPQKSTWRAYTAE (SEQ ID NO:51).

In an embodiment, the monovalent antibody binds to GAL-9 and comprises one of the following combinations of CDRs:
a CDR1 comprising or consisting of the sequence DTSGEYS (SEQ ID NO:56); a CDR2 comprising or consisting of the sequence DQGGIEI (SEQ ID NO:57); and a CDR3 comprising or consisting of the sequence HTFYDMPDGYRQ (SEQ ID NO:58);
a CDR1 comprising or consisting of the sequence STSADST (SEQ ID NO:59); a CDR2 comprising or consisting of the sequence ENNGADR (SEQ ID NO:60); and a CDR3 comprising or consisting of the sequence YWKIAYTKQYEFL (SEQ ID NO:61);
a CDR1 comprising or consisting of the sequence DGSGWDI (SEQ ID NO:62); a CDR2 comprising or consisting of the sequence RHDTTYS (SEQ ID NO:63); and a CDR3 comprising or consisting of the sequence TIPASWNKS (SEQ ID NO:64); or
a CDR1 comprising or consisting of the sequence GTWRWTI (SEQ ID NO:65); a CDR2 comprising or consisting of the sequence RTNSEYQ (SEQ ID NO:66); and a CDR3 comprising or consisting of the sequence EPTAVWADKGQEMQS (SEQ ID NO:67).

In an embodiment, the monovalent antibody comprises: (i) a framework region (FR) 1 comprising or consisting of an amino acid sequence having at least 50%, 60%, 70%, 75%, 80%, 85%, 90% or 95% identity with the sequence MAEVQLQASGGGFVQPGGSLRLSCAASG (SEQ ID NO: 52); (ii) a FR2 comprising or consisting of an amino acid sequence having at least 50%, 60%, 70%, 75%, 80%, 85%, 90% or 95% identity with the sequence MGWFRQAPGKEREFVSAIS (SEQ ID NO:53); (iii) a FR3 comprising or consisting of an amino acid sequence having at least 50%, 60%, 70%, 75%, 80%, 85%, 90% or 95% identity with the sequence MGWFRQAPGKEREFVSAIS (SEQ ID NO:54); (iv) a FR4 comprising or consisting of an amino acid sequence having at least 50%, 60%, 70%, 75%, 80%, 85%, 90% or 95% identity with the sequence YWGQGTQVTVSS (SEQ ID NO:55); or (v) any combination of (i) to (iv).

In an embodiment, the monovalent antibody comprises: (i) a framework region (FR) 1 comprising or consisting of the sequence MAEVQLQASGGGFVQPGGSLRLSCAASG (SEQ ID NO: 52); (ii) a FR2 comprising or consisting of the sequence MGWFRQAPGKEREFVSAIS (SEQ ID NO: 53); (iii) of the sequence a FR3 comprising or consisting YYADSVKGRFTISRDNSKNTVYLQMNSLRAEDTATYYCA (SEQ ID NO:54); (iv) a FR4 comprising or consisting of the sequence YWGQGTQVTVSS (SEQ ID NO:55); or (v) any combination of (i) to (iv).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N1 depicted at FIG. 4 (SEQ ID NO:1). In a further embodiment, the monovalent antibody comprises or consists of the sequence of the antibody G13N1 depicted at FIG. 4 (SEQ ID NO:1).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N2 depicted at FIG. 4 (SEQ ID NO:2). In a further embodiment, the monovalent antibody comprises or consists of the sequence of the antibody G13N2 depicted at FIG. 4 (SEQ ID NO:2).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N3 depicted at FIG. 4 (SEQ ID NO:3). In a further embodiment, the monovalent antibody comprises or consists of the sequence of the antibody G13N3 depicted at FIG. 4 (SEQ ID NO:3).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N4 depicted at FIG. 4 (SEQ ID NO:4). In a further embodiment, the monovalent antibody comprises or consists of the sequence of the antibody G13N4 depicted at FIG. 4 (SEQ ID NO:4).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N5 depicted at FIG. 4 (SEQ ID NO:5). In a further embodiment, the monovalent antibody comprises or consists of the sequence of the antibody G13N5 depicted at FIG. 4 (SEQ ID NO:5).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N6 depicted at FIG. 4 (SEQ ID NO:6). In a further embodiment, the monovalent antibody comprises or consists of the sequence of the antibody G13N6 depicted at FIG. 4 (SEQ ID NO:6).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N7 depicted at FIG. 4 (SEQ ID NO:7). In a further embodiment, the monovalent antibody comprises or consists of the sequence of the antibody G13N7 depicted at FIG. 4 (SEQ ID NO:7).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N8 depicted at FIG. 4 (SEQ ID NO:8). In a further embodiment, the monovalent antibody comprises or consists of the sequence of the antibody G13N8 depicted at FIG. 4 (SEQ ID NO:8).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N9 depicted at FIG. 4 (SEQ ID NO:9). In a further embodiment, the monovalent antibody comprises or consists of the sequence of the antibody G13N9 depicted at FIG. 4 (SEQ ID NO:9).

In an embodiment, the monovalent antibody binds to GAL-13 and comprises or consists of an amino acid sequence having at least 80%, 85%, 90%, 95%, 96%, 97%, 98% or 99% identity with the sequence of the antibody G13N10 depicted at FIG. 4 (SEQ ID NO:10). In a further embodiment, the monovalent antibody comprises or consists of the sequence of the antibody G13N10 depicted at FIG. 4 (SEQ ID NO:10).

In an embodiment, the monovalent antibody binds to GAL-9 and comprises or consists of an amino acid sequence having at least 80%, 85%, 90%, 95%, 96%, 97%, 98% or 99% identity with the sequence of the antibody G9N1 depicted at FIG. 16 (SEQ ID NO:12). In a further embodiment, the monovalent antibody comprises or consists of the sequence of the antibody G9N1 depicted at FIG. 16 (SEQ ID NO:12).

In an embodiment, the monovalent antibody binds to GAL-9 and comprises or consists of an amino acid sequence having at least 80%, 85%, 90%, 95%, 96%, 97%, 98% or 99% identity with the sequence of the antibody G9N2 depicted at FIG. 16 (SEQ ID NO:13). In a further embodiment, the monovalent antibody comprises or consists of the sequence of the antibody G9N2 depicted at FIG. 16 (SEQ ID NO:13).

In an embodiment, the monovalent antibody binds to GAL-9 and comprises or consists of an amino acid sequence having at least 80%, 85%, 90%, 95%, 96%, 97%, 98% or 99% identity with the sequence of the antibody G9N3 depicted at FIG. 16 (SEQ ID NO:14). In a further embodiment, the monovalent antibody comprises or consists of the sequence of the antibody G9N3 depicted at FIG. 16 (SEQ ID NO:14).

In an embodiment, the monovalent antibody binds to GAL-9 and comprises or consists of an amino acid sequence having at least 80%, 85%, 90%, 95%, 96%, 97%, 98% or 99% identity with the sequence of the antibody G9N4 depicted at FIG. 16 (SEQ ID NO:15). In a further embodiment, the monovalent antibody comprises or consists of the sequence of the antibody G9N4 depicted at FIG. 16 (SEQ ID NO:15).

In another embodiment, the above-noted monovalent antibody binding to GAL-13 also binds to GAL-16.

Variations in the monovalent antibody described herein, can be made, for example, using any of the techniques and guidelines for conservative and non-conservative mutations set forth, for instance, in U.S. Pat. No. 5,364,934. Variations may be a substitution, deletion or insertion of one or more codons encoding the monovalent antibody that results in a change in the amino acid sequence as compared with the native sequence antibody. Optionally the variation is by substitution of at least one amino acid with any other amino acid (including naturally occurring amino acids as well as amino acid analogs) in one or more of the domains of the monovalent antibody. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting monovalent antibody variants for activity exhibited by the “native” (or reference) monovalent antibody.

“Identity” refers to sequence identity between two polypeptides. Identity can be determined by comparing each position in the aligned sequences. Methods of determining percent identity are known in the art, and several tools and programs are available to align amino acid sequences and determine a percentage of identity including EMBOSS Needle, ClustalW, SIM, DIALIGN, etc. As used herein, a given percentage of identity with respect to a specified subject sequence, or a specified portion thereof, may be defined as the percentage of amino acids in the candidate derivative sequence identical with the amino acids in the subject sequence (or specified portion thereof), after aligning the sequences and introducing gaps, if necessary to achieve the maximum percent sequence identity, as generated by the Smith Waterman algorithm (Smith & Waterman, J. Mol. Biol. 147 147:195-7 (1981)) using the BLOSUM substitution matrices (Henikoff & Henikoff, Proc. Natl. Acad. Sci. USA 89:10915-9 (1992)) as similarity measures. A “% identity value” is determined by the number of matching identical amino acids divided by the sequence length for which the percent identity is being reported.

In an embodiment, the monovalent antibodies of the present disclosure may be subjected to in vitro affinity maturation. A library comprising variants of the monovalent antibodies disclosed herein may be generated and screened to identity monovalent antibodies having improved affinity and/or specificity for the target antigen (GAL-9 or 13/16). Thus, in another aspect, the present disclosure provides a method for identifying affinity-matured monovalent antibodies specific for GAL-9 or 13 comprising: (i) generating a library of test monovalent antibodies, wherein said test monovalent antibodies comprises one or more mutations (point mutations, substitutions) relative to the parent monovalent antibody disclosed herein (FIGS. 4 and 16); (ii) selecting the monovalent antibodies that binds to GAL-13/16 or GAL-9 with higher affinity than the parent monovalent antibody, thereby identifying affinity-matured monovalent antibodies. In an embodiment, the one or more mutations is in one or more of the CDRs disclosed herein. In an embodiment, the test monovalent antibody comprises 15 mutations or less relative to the parent monovalent antibody. In an embodiment, the test monovalent antibody comprises 10 mutations or less relative to the parent monovalent antibody. In embodiments, the test monovalent antibody comprises 9, 8, 7, 6, or 5 mutations or less relative to the parent monovalent antibody. In an embodiment, the affinity of the affinity-matured monovalent antibody for GAL-13 or GAL-9 is at least 2-fold that of the parent monovalent antibody. In embodiments, the affinity of the affinity-matured monovalent antibody for GAL-13/16 or GAL-9 is at least 5-, 10-, 20-, 50- or 100-fold that of the parent monovalent antibody.

Modifications to the C or N-terminal V_{H} framework sequence may be made to the monovalent antibodies of the disclosure to improve their properties. For example, the V_{H} domain may comprise C or N-terminal extensions or deletions. C-terminal extensions can be added to the C terminal end of a V_{H} domain.

In one embodiment, the monovalent antibodies of the disclosure comprise C-terminal extensions or deletions of from 1 to 50, or more residues, for example 1 to 25, e.g., 1, 2, 3, 4, 5, 6, 7, 8, 9 or 10 additional amino acids.

Additional C or N-terminal residues can be linkers that are used to conjugate the monovalent antibodies of the disclosure to another moiety, or tags that facilitate the detection of the molecule (GAL-13/16 or GAL-9). Such tags are well known in the art and include for example polyhistidine tags (His-tags), polyarginine tags, polyaspartate tags, polycysteine tags, polyphenylalanine tags, glutathione S-transferase (GST) tags, Maltose binding protein (MBP) tags, calmodulin binding peptide (CBP) tags, Streptavidin/Biotin-based tags, HaloTag®, Profinity eXact® tags, epitope tags (such as FLAG, hemagglutinin (HA), HSV, S/S1, c-myc, KT3, T7, V5, E2, and Glu-Glu epitope tags), reporter tags such as β-galactosidase (β-gal), alkaline phosphatase (AP), chloramphenicol acetyl transferase (CAT), and horseradish peroxidase (HRP) tags (see, e.g., Kimple et al., Curr Protoc Protein Sci. 2013; 73: Unit-9.9).

The monovalent antibody according to the present disclosure may comprise at least one constant domain, e.g., a constant domain of a light and/or heavy chain, or a fragment thereof. For example, the monovalent antibody may comprise a Fragment crystallizable (Fc) region or domain of the constant heavy chain of an antibody. The Fc fragment may comprise two or three constant domains, e.g., a CH₂ domain and CH₃ domain. The Fc region may be obtained from a human IgG1, a human IgG4, or a variant of a human IgG1 or IgG4 having up to ten amino acid modifications, for example. In an embodiment, the Fc fragment comprises or consists of the CH₂ domain and CH₃ domain of a human antibody, preferably a human IgG such as IgG1. The presence of an Fc domain on the monovalent antibody may promote antibody-mediated activities such as complement-dependent cytotoxicity (CDC), antibody-dependent cellular cytotoxicity (ADCC) and/or antibody-dependent cellular phagocytosis (ADCP), i.e., the cytotoxic killing or phagocytosis of cells bound by the monovalent antibody (e.g., GAL-13- or GAL-9-expressing tumor cells).

The monovalent antibody according to the present disclosure may be linked to other function or non-functional groups, for example the monovalent antibody may be conjugated to a label (e.g., a biotin label, a fluorescent label, an enzyme label, a coenzyme label, a chemiluminescent label), a nanoparticle, a drug (e.g., a chemotherapeutic agent, an anti-inflammatory drug), a peptide, a nucleic acid, a toxin, an enzyme, a radioisotope, a half-life extending moiety (e.g., PEGylation, using a serum albumin protein), a therapeutic molecule or any other chemical moiety. The monovalent antibody may be used to target GAL-13/16- or GAL-9-expressing cells (e.g., cancer cells), or may be used to detect GAL-13/16 or GAL-9 and/or cells expressing GAL-13/16 or GAL-9, in diagnostic, prognostic, disease monitoring, therapeutic and medical imaging applications.

In an embodiment, the monovalent antibody according to the present disclosure is conjugated to one or more therapeutic or active agents (e.g., a drug), and thus may also be used therapeutically to deliver the therapeutic agent(s) (e.g., anti-tumor agent or any other agent useful for the treatment of the disease or condition or for relieving one or more symptoms) into a cell or tissue, such as a tumor. The monovalent antibody according to the present disclosure may also be conjugated to agent capable of inducing the degradation of the target galectin (e.g., bringing the sdAb-galectin complex to the ubiquitination or lysosomal machinery), for example a E3 ubiquitin ligase binding moiety (to produce a PROteolysis TArgeting Chimera, PROTAC) or a lysosome-targeting receptor (LTR) binding moiety (to produce a LYsosomal TArgeting Chimera, LYTAC). In an embodiment, the sdAb is linked to the E3 ubiquitin ligase binding moiety or LTR binding moiety through a linker (e.g., an amino acid linker comprising aminohexanoic acid (Ahx), glycine, and serine, or a polyethylene glycol linker). Examples of E3 ubiquitin ligase binding moiety (or E3 ligands) are disclosed in the online database of PROTACs (http://cadd.zju.edu.cn/protacdb/, Weng et al., Nucleic Acids Res. 2023 Jan. 6; 51 (D1): D1367-D1372. doi: 10.1093/nar/gkac946).

Any method known in the art for conjugating the monovalent antibody thereof to another moiety (e.g., detectable moiety, active agent, peptide) may be employed (Hermanson, Bioconjugate Techniques, 3^{rd} edition, 2013, Academic Press, Inc., San Diego).

SdAbs may be produced in various expression systems including E. coli, yeasts, or filamentous fungi (see, for example, Harmsen and De Haard, Appl Microbiol Biotechnol. 2007 November; 77(1): 13-22).

A further aspect of the present disclosure provides nucleic acids encoding the monovalent antibody according to the present disclosure. For example, the isolated nucleic acid may be a synthetic DNA (e.g., cDNA), an mRNA (naturally or non-naturally occurring mRNA). The nucleic acid may be inserted within a plasmid, vector, or transcription or expression cassette. The nucleic acids encoding the monovalent antibody according to the present disclosure may be made and the expressed monovalent antibodies may be tested using conventional techniques well known in the art. In some embodiments, the nucleic acid encoding the monovalent antibody described herein can be maintained in the vector in a host cell. In some embodiments, the nucleic acid is an expression vector. In some embodiments, the nucleic acid sequence encoding the monovalent antibody can be maintained in the vector in a host cell. In embodiment, the nucleic acid(s) (DNA, mRNA) encoding the monovalent antibody described herein of the disclosure is comprised within a vesicle such as lipid nanoparticles (e.g., liposomes) or any other suitable vehicle. In an embodiment, the nucleic acid is an mRNA and is encapsulated into nanoparticulate delivery vehicles (see, e.g., Van Hoecke and Roose (2019) How mRNA therapeutics are entering the monoclonal antibody field, J. Transl. Med. 17, 54. https://doi.org/10.1186/s12967-019-1804-8; Sanz and Álvarez-Vallina (2021) Engineered mRNA and the Rise of Next-Generation Antibodies, Antibodies 10 (4): 37. https://doi.org/10.3390/antib10040037).

In another aspect, the present disclosure provides a cell, for example a recombinant host cell, comprising the above-noted nucleic acids and expressing the monovalent antibody according to the present disclosure. Methods of preparing monovalent antibodies comprise expressing the encoding nucleic acid(s) in a host cell under conditions to produce the antibodies, and recovering the antibodies. The process of recovering the antibodies may comprise isolation and/or purification of the antibodies. The method of production may comprise formulating the antibodies into a composition including at least one additional component, such as a pharmaceutically acceptable excipient.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. Coli; mammalian cell lines CHO, HEK 293 and COS; the insect cell line Sf9; the fungal cell Saccharomyces cerevisiae, plant cells, or algae cells.

In another embodiment, the host cell is an immune cell. The anti-GAL-13/16 or anti-GAL-9 monovalent antibody described herein may be used as a chimeric antigen receptor (CAR) to produce CAR T cells, CAR NK cells, etc. CAR combines a ligand-binding domain (e.g., antibody or antibody fragment) that provides specificity for a desired antigen (e.g., GAL-13/16 or GAL-9) with an activating intracellular domain (or signal transducing domain) portion, such as a T cell or NK cell activating domain, providing a primary activation signal. SdAbs capable of binding to molecules expressed by tumor cells are commonly used as CAR. Thus, in another aspect, the present disclosure provides a host cell, preferably an immune cell such as a T cell or NK cell, expressing the monovalent antibody described herein.

The CAR of the present disclosure may also comprise a transmembrane domain which spans the membrane. The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, CD28, CD3-epsilon, CD45, CD4, CD5, CD8, CD9, CD16, CD22, CD33, CD37, CD64, CD80, CD86, CD134, CD137, ICOS, CD154, or a GITR can be used. The artificially designed transmembrane domain is a polypeptide mainly comprising hydrophobic residues such as leucine and valine. It is preferable that a triplet of phenylalanine, tryptophan and valine is found at each end of the synthetic transmembrane domain. In preferred embodiments, the transmembrane domain is derived from CD28 or CD8, which give good receptor stability.

Preferred examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for Syk/ZAP70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d.

The CAR of the present disclosure may also comprise one or more co-stimulatory domains such as human CD28, 4-1BB (CD137), ICOS-1, CD27, OX40 (CD137), DAP10, and GITR (AITR). In embodiment, the CAR is a third generation and comprises two co-stimulating domains such as CD28 and 4-1BB.

The CAR of the present disclosure may also comprise a signal peptide N-terminal to the anti-GAL-13/16 or anti-GAL-9 monovalent antibody described herein so that when the CAR is expressed inside a cell, such as a T-cell, the nascent protein is directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. As an example, the signal peptide may derive from human CD8 or GM-CSF, or a variant thereof having 1 or 2 amino acid mutations provided that the signal peptide still functions to cause cell surface expression of the CAR.

The CAR of the present disclosure may comprise a spacer sequence as a hinge to connect the anti-GAL-13/16 or anti-GAL-9 monovalent antibody described herein with the transmembrane domain and spatially separate antigen binding domain from the endodomain. A flexible spacer allows to the binding domain to orient in different directions to enable its binding to the desired antigen (e.g., GAL-13/16 or GAL-9). The spacer sequence may, for example, comprise an IgG1 Fc region, an IgG1 hinge or a CD8 stalk, or a combination thereof.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Introducing such nucleic acids into a host cell can be accomplished using techniques well known in the art. For eukaryotic cells, suitable techniques may include calcium phosphate transfection, DEAE-Dextran, electroporation, liposome-mediated transfection, and transduction using retroviruses or other viruses, for example. For bacterial cells, suitable techniques may include calcium chloride transformation, electroporation, and transfection using bacteriophage. The introduction may be followed by causing or allowing expression from the nucleic acid, e.g., by culturing host cells under conditions for expression of the gene. In one embodiment, the nucleic acid of the invention is integrated into the genome, e.g., chromosome, of the host cell. Integration may be promoted by inclusion of sequences that promote recombination with the genome, in accordance with standard techniques.

Systems for cloning and expression of a polypeptide in a variety of different host cells are well known. Suitable host cells include bacteria, mammalian cells, plant cells, insect cells, fungi, yeast and transgenic plants and animals. Mammalian cell lines available in the art for expression of a heterologous polypeptide include Chinese hamster ovary (CHO) cells, Hela cells, baby hamster kidney cells, mouse melanoma cells, rat myeloma cells, human embryonic kidney cells, e.g., HEK293 cells, human embryonic retina cells, and many others. The expression of antibodies and antibody fragments in prokaryotic cells, such as E. coli, is well established in the art. For a review, see for example, Pliickthun, Bio/Technology 9:545-551 (1991). Expression in cultured eukaryotic cells is also available to those skilled in the art, as reviewed in Andersen et al. (2002) Curr. Opin. Biotechnol. 13:117-23, for example.

In another aspect, the present disclosure provides a composition (e.g., a pharmaceutical composition) comprising the above-mentioned monovalent antibody. In an embodiment, the composition further comprises one or more pharmaceutically acceptable carriers, excipients, and/or diluents.

As used herein, “pharmaceutically acceptable” (or “biologically acceptable”) refers to materials characterized by the absence of (or limited) toxic or adverse biological effects in vivo. It refers to those compounds, compositions, and/or dosage forms which are, within the scope of sound medical judgment, suitable for use in contact with the biological fluids and/or tissues and/or organs of a subject (e.g., human, animal) without excessive toxicity, irritation, allergic response, or other problem or complication, commensurate with a reasonable benefit/risk ratio.

The term “pharmaceutically acceptable carriers, excipient, and/or diluents” refers to additives commonly used in the preparation of pharmaceutical compositions and includes, for example, solvents, dispersion media, saline solutions, surfactants, solubilizing agents, lubricants, emulsifiers, coatings, antibacterial and antifungal agents, chelating agents, pH-modifiers, soothing agents, buffers, reducing agents, antioxidants, isotonic agents, absorption delaying agents or the like. Such compositions may be prepared in a manner well known in the pharmaceutical art by mixing the antibody having a suitable degree of purity with one or more optional pharmaceutically acceptable carriers or excipients (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The carrier/excipient can be suitable for administration of the antibody or conjugate thereof by any conventional administration route, for example, for oral, intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. In an embodiment, the carrier/excipient is adapted for administration of the antibody or conjugate thereof by the intravenous or subcutaneous route. In an embodiment, the carriers/excipients are adapted for administration of the antibody or conjugate thereof by the intravenous route. In another embodiment, the carriers/excipients are adapted for administration of the antibody or conjugate thereof by the subcutaneous route.

The composition may also comprise one or more additional active agents for the treatment the targeted disease/condition or for the management of one or more symptoms of the targeted disease/condition (e.g., pain killers, anti-nausea agents, etc.), as described in more detail below.

The monovalent antibody, conjugate or composition of the present disclosure may be used to inhibit any biological, physiological and/or pathological process that involves GAL-13/16 or GAL-9 activity.

In another aspect, the present disclosure provides a method (in vitro or in vivo) for binding to GAL-13/16 or GAL-9, said method comprising contacting said GAL-13/16 or GAL-9 with the monovalent antibody, conjugate or composition described herein. In an embodiment, the above-mentioned method is for binding to GAL-13/16 or GAL-9 in a cell or in the extracellular space (since galectins may be released by cells via a non-classical secretory pathway). The present disclosure also provides the use of the monovalent antibody, conjugate or composition described herein for binding to GAL-13/16 or GAL-9. The present disclosure also provides the use of the monovalent antibody, conjugate or composition described herein for the manufacture of a medicament for binding to GAL-13/16 or GAL-9. The method or use for binding to GAL-13/16 or GAL-9 may be useful in diagnostic, disease monitoring, prognostic or therapeutic application, notably to detect GAL-13/16 or GAL-9, to identify and/or target GAL-13/16- or GAL-9-expressing cells (e.g., by CAR cells), to deliver molecules (e.g., cytotoxic agents) to GAL-13/16- or GAL-9-expressing cells.

In another aspect, the present disclosure provides a method (in vitro or in vivo) for inhibiting the activity of GAL-13/16 or GAL-9, said method comprising contacting said GAL-13/16 or GAL-9 with the monovalent antibody, conjugate or composition described herein. In an embodiment, the above-mentioned method is for inhibiting the dimerization of GAL-13/16 or GAL-9 in a cell or in the extracellular space. The present disclosure also provides the use of the monovalent antibody, conjugate or composition described herein for inhibiting the activity of GAL-13/16 or GAL-9. The present disclosure also provides the use of the monovalent antibody, conjugate or composition described herein for the manufacture of a medicament for inhibiting the activity of GAL-13/16 or GAL-9.

Recombinant GAL-13 has been shown to kill certain types of cells, such as Jurkat T cells, monocytes and human peripheral T cells, suggesting that GAL-13 has immunosuppressive properties. GAL-13 is strongly expressed in the syncytiotrophoblast at the lining of the maternal-fetal interface. GAL-13 has been shown to induce the apoptosis of pre-activated T lymphocytes and to increase IL-1a and IL-6 secretion from peripheral blood mononuclear cells (PBMCs) in pregnant women.

In another aspect, the present disclosure provides a method for inhibiting GAL-13-mediated apoptosis in a cell, said method comprising contacting said cell with the anti-GAL-13 monovalent antibody, conjugate or composition described herein. The present disclosure also provides the use of the anti-GAL-13 monovalent antibody, conjugate or composition described herein for inhibiting GAL-13-mediated apoptosis in a cell. The present disclosure also provides the use of the anti-GAL-13 monovalent antibody, conjugate or composition described herein for the manufacture of a medicament for inhibiting GAL-13-mediated apoptosis in a cell. In an embodiment, the above-mentioned cell is an immune cell, such as a T lymphocyte or a monocyte. In another aspect, the present disclosure provides a method for inhibiting GAL-13-mediated immunosuppression in a subject, the method comprising administering to the subject an effective amount of the anti-GAL-13 monovalent antibody, conjugate or composition described herein. The present disclosure also provides the use of the anti-GAL-13 monovalent antibody, conjugate or composition described herein for inhibiting GAL-13-mediated immunosuppression in a subject. The present disclosure also provides the use of the anti-GAL-13 monovalent antibody, conjugate or composition described herein for the manufacture of a medicament for inhibiting GAL-13-mediated immunosuppression in a subject. In an embodiment, the subject suffers from a GAL-13-expressing cancer. In an embodiment, the monovalent antibody or conjugate reduces or inhibits the binding of extracellular GAL-13 to glycoreceptors expressed by infiltrated immune cells.

GAL-13 is also expressed in the spleen, kidney, and bladder, as well as in liver adenocarcinoma, neurogenic tumors, and malignant melanoma (Fuselier et al., Cells 2023, 12, 437).

In another aspect, the present disclosure provides a method for treating a GAL-13-expressing cancer (e.g., inhibiting tumor growth and/or metastasis) in a subject, said method comprising administering to said subject an effective amount of the anti-GAL-13 monovalent antibody, conjugate or composition described herein. The present disclosure also provides the use of the anti-GAL-13 monovalent antibody, conjugate or composition described herein for treating a GAL-13-expressing cancer in a subject. The present disclosure also provides the use of the anti-GAL-13 monovalent antibody, conjugate or composition described herein for the manufacture of a medicament for treating a GAL-13-expressing cancer in a subject.

In another aspect, the present disclosure provides a method for detecting, diagnosing and/or monitoring the progression of a GAL-13-expressing cancer (e.g., monitoring tumor size and/or metastasis) in a subject, said method comprising administering to said subject an effective amount of the monovalent antibody or the composition described herein. The present disclosure also provides the use of the anti-GAL-13 monovalent antibody or the composition described herein for detecting, diagnosing and/or monitoring the progression of a GAL-13-expressing cancer in a subject. The present disclosure also provides the use of the anti-GAL-13 monovalent antibody or the composition described herein for the manufacture of an agent for detecting, diagnosing and/or monitoring the progression of a GAL-13-expressing cancer in a subject.

In an embodiment, the GAL-13-expressing cancer is of epithelial origin. In another embodiment, the GAL-13-expressing cancer is liver adenocarcinoma or a malignant melanoma. In another embodiment, the cancer is a cancer of neural cells (neurogenic tumor), for example a medulloblastoma, glioblastoma or neuroblastoma.

GAL-16 is expressed in the placenta as well as in brain tissues and the retina. Like other galectins such as GAL-13, GAL-16 can induce apoptosis of CD3+ T cells. Alterations in the expression or mutations of GAL-16 has been detected in diseases and conditions such as Alzheimer's Disease (AD), psychiatric disorders, chronic myeloid leukemia (CML), breast cancer, large B-cell lymphoma and type 2 diabetes (Kaminker J D and Timoshenko A V. Biomolecules. 2021; 11 (12): 1909).

In another aspect, the present disclosure provides a method for inhibiting GAL-16-mediated apoptosis in a cell, said method comprising contacting said cell with the anti-GAL-13/16 monovalent antibody or the composition described herein. The present disclosure also provides the use of the anti-GAL-13/16 monovalent antibody or the composition described herein for inhibiting GAL-16-mediated apoptosis in a cell. The present disclosure also provides the use of the anti-GAL-13/16 monovalent antibody, conjugate or composition described herein for the manufacture of a medicament for inhibiting GAL-16-mediated apoptosis in a cell. In an embodiment, the above-mentioned cell is an immune cell, such as a T lymphocyte or a monocyte. In another aspect, the present disclosure provides a method for inhibiting GAL-16-mediated immunosuppression in a subject, said method comprising administering to said subject an effective amount of the anti-GAL-13/16 monovalent antibody, conjugate or composition described herein. The present disclosure also provides the use of the anti-GAL-13/16 monovalent antibody, conjugate or composition described herein for inhibiting GAL-16-mediated immunosuppression in a subject. The present disclosure also provides the use of the anti-GAL-13/16 monovalent antibody, conjugate or composition described herein for the manufacture of a medicament for inhibiting GAL-16-mediated immunosuppression in a subject. In an embodiment, the subject suffers from a GAL-16-expressing cancer. In an embodiment, the monovalent antibody or conjugate reduces or inhibits the binding of extracellular GAL-16 to glycoreceptors expressed by infiltrated immune cells.

GAL-16 is also expressed in certain cancers such as chronic myeloid leukemia (CML), breast cancer, large B-cell lymphoma (Fuselier et al., Cells 2023, 12, 437).

In another aspect, the present disclosure provides a method for treating a GAL-16-expressing cancer (e.g., inhibiting tumor growth and/or metastasis) in a subject, said method comprising administering to said subject an effective amount of the anti-GAL-13/16 monovalent antibody, conjugate or composition described herein. The present disclosure also provides the use of the anti-GAL-13/16 monovalent antibody, conjugate or composition described herein for treating a GAL-16-expressing cancer in a subject. The present disclosure also provides the use of the anti-GAL-13/16 monovalent antibody, conjugate or composition described herein for the manufacture of a medicament for treating a GAL-16-expressing cancer in a subject.

In another aspect, the present disclosure provides a method for detecting, diagnosing and/or monitoring the progression of a GAL-16-expressing cancer (e.g., monitoring tumor size and/or metastasis) in a subject, said method comprising administering to said subject an effective amount of the monovalent antibody, conjugate or composition described herein. The present disclosure also provides the use of the anti-GAL-13/16 monovalent antibody, conjugate or composition described herein for detecting, diagnosing and/or monitoring the progression of a GAL-16-expressing cancer in a subject. The present disclosure also provides the use of the anti-GAL-13/16 monovalent antibody, conjugate or composition described herein for the manufacture of an agent for detecting, diagnosing and/or monitoring the progression of a GAL-16-expressing cancer in a subject.

In an embodiment, the GAL-16-expressing cancer is chronic myeloid leukemia (CML), breast cancer, or B-cell lymphoma.

GAL-9 is highly expressed in rheumatoid arthritis (RA) synovial fluid (SF) and synovial tissue (ST) compared with normal or osteoarthritic ST (Seki M, et al., Arthritis Rheum. 2007; 56 (12): 3968-76), suggesting a role for GAL-9 in RA. GAL-9 has been shown to mediate angiogenesis, increase monocyte migration in vitro, and induces acute inflammatory arthritis in mice, suggesting a role for GAL-9 in angiogenesis, joint inflammation, and possibly other inflammatory diseases (O'Brien et al., Arthritis Research & Therapy volume 20, Article number: 31 (2018).

In another aspect, the present disclosure provides a method for inhibiting GAL-9-mediated inflammation in a tissue or organ, said method comprising contacting said tissue or organ with the anti-GAL-9 monovalent antibody, conjugate or composition described herein. The present disclosure also provides the use of the anti-GAL-9 monovalent antibody, conjugate or composition described herein for inhibiting GAL-9-mediated inflammation in a tissue or organ. The present disclosure also provides the use of the anti-GAL-9 monovalent antibody, conjugate or composition described herein for the manufacture of a medicament for inhibiting GAL-9-mediated inflammation in a tissue or organ. In an embodiment, the tissue is a joint.

In another aspect, the present disclosure provides a method for treating an inflammatory disease associated with GAL-9 activity in a subject, said method comprising administering the anti-GAL-9 monovalent antibody or the composition described herein to the subject. The present disclosure also provides the use of the anti-GAL-9 monovalent antibody or the composition described herein for treating an inflammatory disease associated with GAL-9 activity in a subject. The present disclosure also provides the use of the anti-GAL-9 monovalent antibody or the composition described herein for the manufacture of a medicament for treating an inflammatory disease associated with GAL-9 activity in a subject. In an embodiment, the inflammatory disease is arthritis, e.g., rheumatoid arthritis.

The expression of GAL-9 has been detected on various hematological malignancies, such as Chronic lymphocytic leukemia (CLL), myelodysplastic syndromes (MDS), Hodgkin and Non-Hodgkin lymphomas, acute myeloid leukemia (AML) or solid tumors, such as melanoma, lung cancer, breast cancer, colon cancer and hepatocellular carcinoma, and high GAL-9 expression has been associated with poor prognosis in multiple human cancers. Interaction of GAL-9 with the T-cell immunoglobulin and mucin-domain containing-3 (TIM-3) receptor (HAVCR2) has been shown to attenuate T-cell expansion and effectors function in tumor microenvironment and chronic infections. Moreover, GAL-9 may contribute to tumorigenesis by tumor cell transformation, cell-cycle regulation, angiogenesis, and cell adhesion.

In another aspect, the present disclosure provides a method for treating a GAL-9-expressing cancer (e.g., inhibiting tumor growth and/or metastasis) in a subject, said method comprising administering to said subject an effective amount of the anti-GAL-9 monovalent antibody, conjugate, or composition described herein. The present disclosure also provides the use of the anti-GAL-9 monovalent antibody, conjugate or composition described herein for treating a GAL-9-expressing cancer in a subject. The present disclosure also provides the use of the anti-GAL-9 monovalent antibody, conjugate or composition described herein for the manufacture of a medicament for treating a GAL-9-expressing cancer in a subject.

In another aspect, the present disclosure provides a method for detecting, diagnosing and/or monitoring the progression of a GAL-9-expressing cancer (e.g., monitoring tumor size and/or metastasis) in a subject, said method comprising administering to said subject an effective amount of the monovalent antibody or the composition described herein. The present disclosure also provides the use of the anti-GAL-9 monovalent antibody or the composition described herein for detecting, diagnosing and/or monitoring the progression of a GAL-9-expressing cancer in a subject. The present disclosure also provides the use of the anti-GAL-9 monovalent antibody or the composition described herein for the manufacture of an agent for detecting, diagnosing and/or monitoring the progression of a GAL-9-expressing cancer in a subject.

In an embodiment, the GAL-9-expressing cancer is a hematological cancer. In a further embodiment, the hematological cancer is chronic lymphocytic leukemia (CLL), myelodysplastic syndromes (MDS), Hodgkin lymphoma, Non-Hodgkin lymphoma, or acute myeloid leukemia (AML). In another embodiment, the GAL-9-expressing cancer is a solid cancer. In a further embodiment, the solid cancer is melanoma, lung cancer, breast cancer, colon cancer or liver cancer (e.g., hepatocellular carcinoma). In an embodiment, the GAL-9-expressing cancer is a poor prognosis cancer.

GAL-9 has also been shown to play a role in angiogenesis (Victor L Thijssen, Arjan W Griffioen. Glycobiology, Volume 24, Issue 10, October 2014, Pages 915-920), and thus the anti-GAL-9 monovalent antibody or the composition described herein may be useful for inhibiting angiogenesis.

The amount of the monovalent antibody which is effective for the above-noted activities/therapeutic uses will depend on several factors including the nature and severity of the disease, the chosen prophylactic/therapeutic regimen, the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 1000 mg/kg of body weight/day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a patient, in the context of the present disclosure should be sufficient to effect/induce a beneficial prophylactic and/or therapeutic response in the patient over time (in the case of a cancer, a decrease in tumor size, inhibition of tumor cell proliferation, increased survival time, etc.). The size of the dose also will be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

In an embodiment, the above-mentioned treatment comprises the use/administration of more than one (i.e., a combination of) active/therapeutic agent, including the above-mentioned monovalent antibody. The combination of prophylactic/therapeutic agents and/or compositions of the present disclosure may be administered or co-administered (e.g., consecutively, simultaneously, at different times) in any conventional dosage form. Co-administration in the context of the present disclosure refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) is used/administered in combination with one or more agent(s) or treatment currently used to prevent or treat the disorder in question, e.g., agents or treatments currently used in the treatment of cancers, such as radiotherapy, chemotherapeutic agents, surgery, or immunotherapies (e.g., checkpoint inhibitors, CAR cells).

In an embodiment, the monovalent antibody described herein is used in combination with one or more chemotherapeutic agents. Examples of chemotherapeutic agents suitable for use in combination with the monovalent antibody described herein include, but are not limited to, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), immunotherapeutic agents (e.g., immune checkpoint inhibitors such as PD-1/PD-L1 inhibitors and CTLA-4 inhibitors, B7-1/B7-2 inhibitors, CAR T cells) and other cancer therapeutic agents recognized in the art. In some embodiments, chemotherapeutic agents for use in combination with the monovalent antibody described herein comprise one or more of adriamycin, colchicine, cyclophosphamide, actinomycin, bleomycin, duanorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., taxol, paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Oxaliplatin, Herceptin®, vinorelbine, Doxil®, capecitabine, Alimta®, Avastin®, Velcade®, Tarceva®, Neulasta®, lapatinib, sorafenib, erlotinib, erbitux, PD-1/PD-L1 inhibitors (e.g., nivolumab, pembrolizumab, atezolizumab), CTLA-4 inhibitors (e.g., Ipilimumab), and derivatives thereof, and the like. In an embodiment, the monovalent antibody or composition comprising same described herein is used in combination with an EGFR or tyrosine kinase targeting agent, for example an EGFR inhibitor (RTK inhibitor). The monovalent antibody or composition comprising same described herein may also be used in combination with one or more additional therapeutic antibodies or antibody fragments, e.g., therapeutic antibodies or antibody fragments used for the treatment of tumors or of one or more of the diseases associated with the activity of GAL-13 or GAL-9 described above.

As used herein, the term “subject” is taken to mean warm blooded animals such as mammals, for example, cats, dogs, mice, guinea pigs, horses, bovine cows, sheep and humans. In an embodiment, the subject is a mammal, and more particularly a human.

### MODE(S) FOR CARRYING OUT THE INVENTION

The present invention is illustrated in further details by the following non-limiting examples.

### Example 1: Materials and Methods

Cell line and reagents. The Jurkat cell line was maintained in RPMI 1640 medium. The culture medium was supplemented with 10% [v/v] fetal bovine serum, 2 mmol/L L-glutamine, 10 mM HEPES buffer, and 1 mM sodium pyruvate. HepG2 cell line was maintained in DMEM 4, 5 g glucose supplemented with 10% [v/v] fetal bovine serum. All cell culture products were purchased from Life Technologies (Burlington, ON, Canada).

Production and purification of recombinant galectins. Codon-optimized cDNA were synthesized and subcloned into the pET-22b (+) vector for production in E. coli BL21 (DE3) cells as previously described [Vladoiu et al., 2015]. Purification of galectin-1, -3, -7 and -13 was carried out using conventional lactose affinity and/or mannose chromatography. GAL-3 and -7 were dialyzed against phosphate-buffered saline (PBS) to remove residual lactose. GAL-1 and -13 were dialyzed against Tris buffer. Galectin-16 purification was carried out from inclusion bodies using a 10% sodium docecyl sulfate (SDS) solution and refolded in 25 mM Tris, 10% glycerol, 1M 2-methyl-2,4-pentanediol for 24 h at room temperature. Recombinant human galectin-9 (Gal-9) was purchased (Biolegend, San Diego, CA, USA).

Generation and production of single chain camelid antibodies. Galectin-specific single chain (VHH) camelid antibodies were obtained following in vitro screening of a non-immune humanized synthetic single-domain antibodies (hsd2Ab) library that contains more than 3×109 clones expressed at the surface of M13 phage [Moutel et al., 2016]. The panning was conducted against immobilized recombinant human GAL-13 or GAL-9 devoid of carbohydrate in its glycan binding site (GBS). Prior to the phage display selection, GAL-13-biotin, GAL-9-biotin and GST-Biotin were bound to streptavidin magnetic beads (Dynabeads® M-280 Streptavidin, Life Technologies) with a 50 nM final concentration of biotinylated protein for the first round and 10 nM final concentration of biotinylated protein for the second and third rounds. The successful binding of the biotinylated proteins on the streptavidin beads was controlled for via SDS-polyacrylamide gel electrophoresis/Western blot using a streptavidin-horseradish peroxidase (HRP) conjugate (Thermo Fischer Scientific, MA, USA). Following three-rounds of phage display, clones were picked randomly and analyzed by non-adsorbed phage ELISA using HRP-conjugated anti-M13 antibody (GE Healthcare, ON, Canada) and a colorimetric substrate (TMB, tetramethylbenzidine, Thermo Fischer, MA, USA). Sequences of positive binders were inserted into the prokaryotic vector pHEN2 (containing 6×His and cMyc tags) for production in E. coli BL21 (DE3) and they purified by conventional immobilized metal ion affinity chromatography. Apoptosis of human T cells. In vitro apoptosis assays were carried out using the standard in vitro human Jurkat T cell model system using fluorescein isothiocyanate (FITC)-labeled annexin V (Biolegend, San Diego, CA, USA) and propidium iodide (PI). Briefly, increasing doses of recombinant Gal-13 were added to Jurkat cells in 1% RPMI 1640 medium. Cells were incubated at 20° C. for 16 h. Mixtures of recombinant GAL-13 and sdAbs were pre-incubated for 1 h at 4° C. before being added to Jurkat cells. Cells were then washed once in PBS and once in binding buffer (0.01 M HEPES, 0.14 M NaCl, 2.5 mM CaCl₂), pH 7.4). For staining, cells were incubated for 15 min with FITC-labeled annexin V in the dark at room temperature. The PI (0.25 μg/mL) stain was added to cells just before analysis by flow cytometry.

ELISA assays. MaxiSorp™ 96-well plates (Thermo Fischer Scientific, MA, USA) were used for all ELISA experiments.

To test the specificity of GAL-13 sdAbs, recombinant human galectins were immobilized in wells at indicated concentrations for 16 h at 4° C. After blocking with PBS containing 10% (v/V) bovine serum albumin (BSA) and 0.05% (v/v) Tween (blocking buffer), ten-fold dilutions of sdAbs were added to each well and incubated for 1 h at room temperature. Binding of sdAbs was revealed using successive incubations with a goat anti-his-tag polyclonal (1/1000) antibody (Bio-Rad, ON, Canada) and a HRP-conjugated donkey anti-goat IgG (1/5000) antibody (R&D Systems, MN, USA). To measure the blocking of GAL-13/asialoetuin (ASF) binding by sdAbs, ASF was immobilized in wells at 10 μg/ml for 16 h at 4° C. in carbonate buffer pH 9.6. After blocking with PBS containing 10% (v/v) BSA and 0.05% (v/v) Tween for 2 h at room temperature, increasing concentrations of GAL-13 were added and incubated for 1 h at room temperature. Recombinant GAL-13 was also mixed with higher doses of sdAbs for 1 h at 4° C. before being added to ASF-immobilized wells. Binding of Gal-13 was revealed using a mouse anti-galectin-13 monoclonal (1/1000) antibody (MyBioSource, CA, USA) and an HRP-conjugated anti-mouse IgG (1/10,000) antibody (Promega, WI, USA). The colorimetric assays were carried out with 3,3′,5,5′-Tetramethylbenzidine (TMB) (Sigma-Aldrich, MO, USA) according to the manufacturer's recommendations.

To test GAL-9 sdAbs, the sdAbs were immobilized in wells at 1 μM for 1 h at room temperature, followed by blocking with PBS containing 10% (v/v) BSA and 0.05% (v/v) Tween. To measure the binding affinities of sdAbs for Gal-9, increasing concentrations of recombinant Gal-9 (Biolegend, San Diego, CA, USA) were added in sdAb-immobilized wells and incubated for 1 h at room temperature. To measure binding affinities of sdAbs to individual GAL-9 domains, 5 μM of recombinant N- and C-terminal domains of human GAL-9 (GAL-9N and GAL-9C respectively) were added to sdAb-immobilized wells and incubated for 1 h at room temperature. The sequences of GAL-9N and GAL-9C are:

GAL-9N (1-148)
(SEQ ID NO: 70)
MAFSGSQAPYLSPAVPFSGTIQGGLQDGLQITVNGTVLSSSGTRFAVNF
QTGFSGNDIAFHFNPRFEDGGYVVCNTRQNGSWGPEERKTHMPFQKGMP
FDLCFLVQSSDFKVMVNGILFVQYFHRVPFHRVDTISVNGSVQLSYISF
Q
GAL-9C (210-355)
(SEQ ID NO: 71)
TPAIPPMMYPHPAYPMPFITTILGGLYPSKSILLSGTVLPSAQRFHINL
CSGNHIAFHLNPRFDENAVVRNTQIDNSWGSEERSLPRKMPFVRGQSFS
VWILCEAHCLKVAVDGQHLFEYYHRLRNLPTINRLEVGGDIQLTHVQT

Binding of Gal-9N and recombinant GAL-9 was revealed using a rabbit anti-human galectin-9 (1/500) antibody (Abcam, ON, Canada) and an HRP-conjugated goat anti-rabbit IgG (1/5000) antibody (Promega, WI, USA). The binding of GAL-9C was revealed using a mouse anti-human GAL-9 clone 9M1-3 (1/200) antibody (Biolegend, San Diego, CA, USA) and an HRP-conjugated goat anti-mouse IgG (1/2000) antibody (Promega, WI, USA). To test the specificity of GAL-9 sdAbs, 5 μM of galectins were added to sdAb-immobilized wells and incubated for 1 h at room temperature. Binding of galectins was revealed through incubation with their respective primary antibodies: rabbit anti-human Gal-9 (1/500) antibody (Abcam, ON, Canada), goat anti-human GAL-7 (1/5000) antibody (R&D Systems, MN, USA), mouse anti-human GAL-1 (1/1000) antibody (Proteintech, IL, USA), mouse anti-human GAL-13 (1/5000) antibody (MyBioSource, CA, USA). Subsequent incubation with the appropriate secondary antibodies followed: goat anti-rabbit IgG (1/10,000) antibody (Promega, WI, USA), donkey HRP-conjugated anti-goat IgG (1/10,000) antibody (R&D Systems, MN, USA), goat anti-mouse IgG (1/10,000) antibody (Promega, WI, USA). The colorimetric assays were carried out with TMB (Sigma-Aldrich, MO, USA) according to the manufacturer's recommendations.

Cell binding assay. Purified recombinant GAL-13 was labeled with Fluorescein Isothiocyanate (FITC) by successive dialyses. Briefly, HepG2 cells were removed with EDTA buffer and counted to have 200 000 cells/tubes. Then, cells were centrifuged at 1000 RPM for 10 min at 4° C., and incubated with 100 μl of PBS or 100 μl of GAL-13-FITC (increasing doses) for 30 min at 4° C. Cells were again centrifuged at 1000 RPM for 10 min at 4° C. and analyzed using BD FACSalibur™ flow cytometry. Binding specificity was performed by incubating HepG2 cells with an excess of unlabeled galectin during 30 min at 4° C. before adding GAL-13-FITC for 30 min. sdAbs' effects were analyzed by incubating Gal-13-FITC with sdAbs for 1 h at 4° C. The mixture was then incubated with HepG2 cells for 30 min at 4° C.

Statistical analysis. Statistical significance was evaluated using the unpaired Student's t-test, Tukey's multiple comparisons test, Dunnett's multiple comparisons test or one-way ANOVA. Results were considered statistically significant at P≤0.05.

### Example 2: Generation and Production of Galectin-13-Specific VHHs Clones

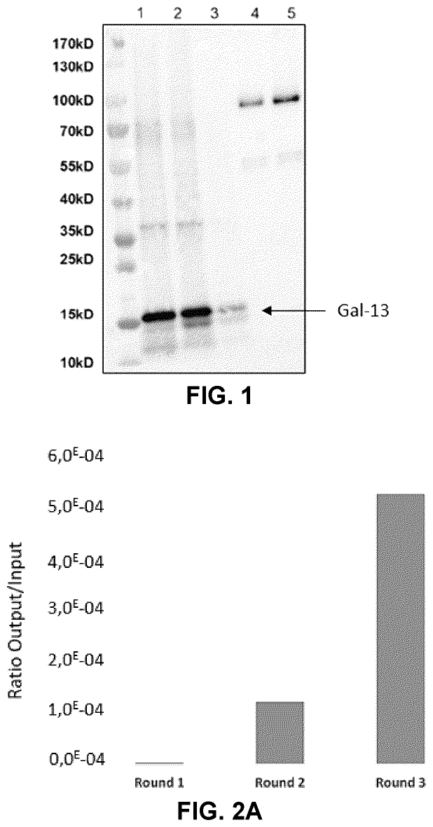

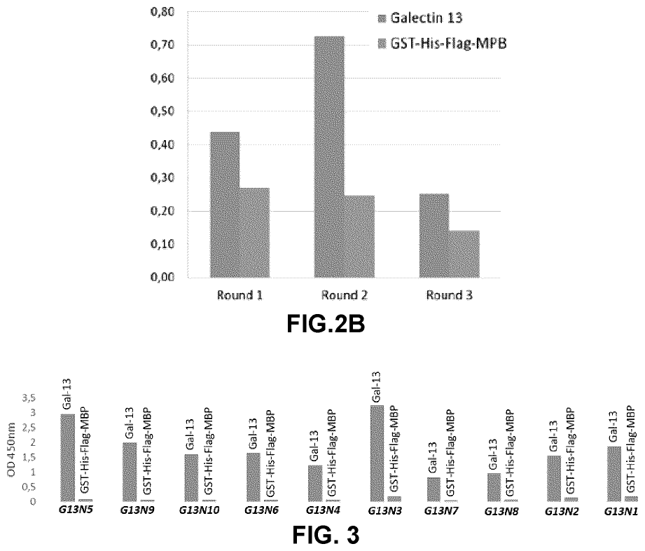

Following an initial pre-screen round of phage display carried out with GST-biotin to deplete the library from unspecific binders, unbound VHHs were incubated with beads conjugated to GAL-13-Biotin (FIG. 1). A total of three rounds of phage panning were performed. Progressive enrichment during the cycle was validated by measuring the output/input ratio (FIG. 2A), and an ELISA on the pool of each round was performed (FIG. 2B). A total of 136 VHHs were then picked and analyzed by ELISA for GAL-13 binders. Ten (10) clones were shown to exhibit strong binding to GAL-13 (G13N1 to G13N10 (FIG. 3). The amino acid sequences of the 10 GAL-13 binding clones are shown in FIG. 4.

### Example 3: Characterization of Galectin-13-Specific VHHs Clones

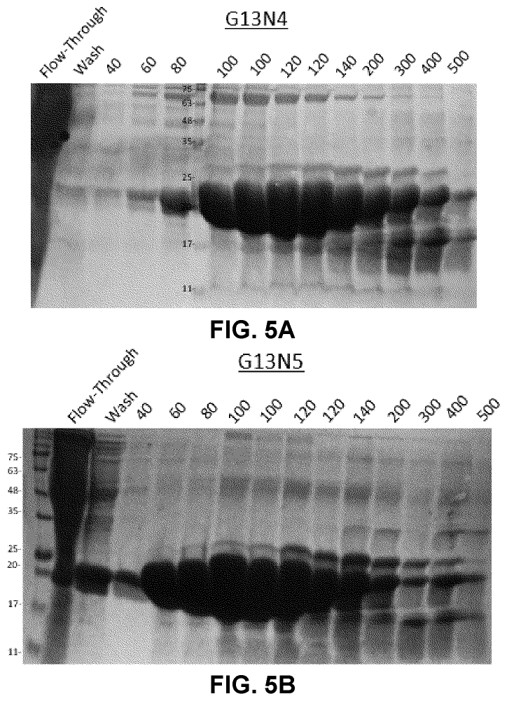

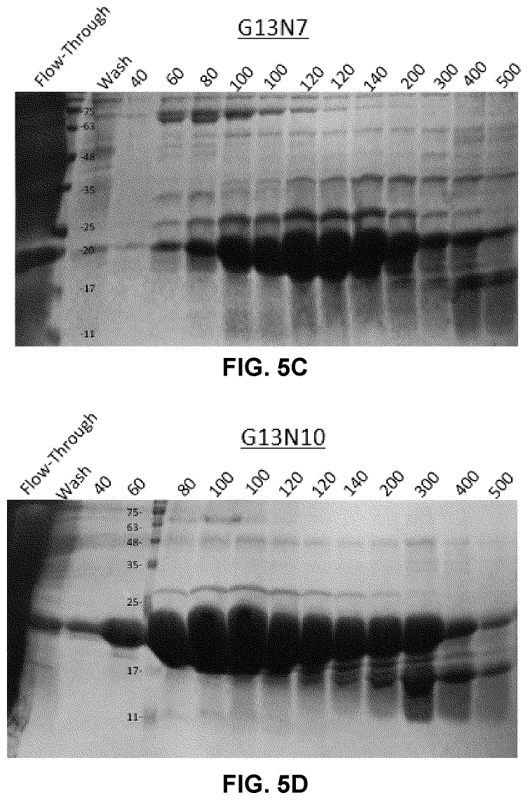

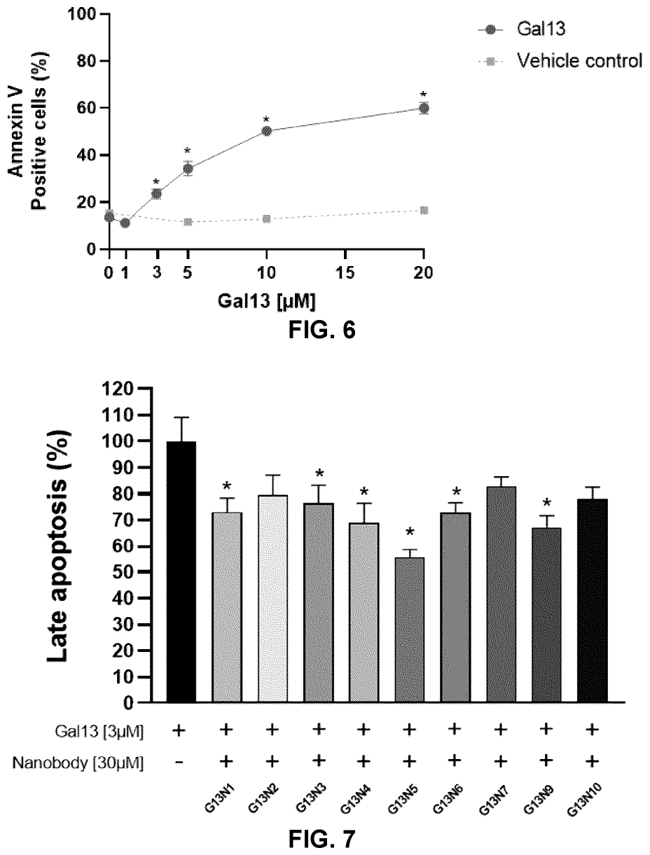

The clones were purified by conventional by metal ion affinity chromatography for characterization. Representative SDS-PAGE analyses for 4 of the clones (G13N4, G13N5, G13N7 and G13N10) are shown in FIG. 5. As shown in FIG. 6, incubation of human T cells with recombinant GAL-13 induces apoptosis. It was next assessed whether the Gal-13 binding sdAbs could prevent GAL-13-mediated apoptosis of T cells. The results showed that all the clones tested led to a certain level of inhibition of galectin-13-induced apoptosis of Jurkat T cells, with sdAbs G13N1 (−27%), G13N3 (−23.5%), G13N4 (−31%), G13N5 (−44%), G13N6 (−27%) and G13N9 (−33%) reaching statistical significance (FIG. 7).

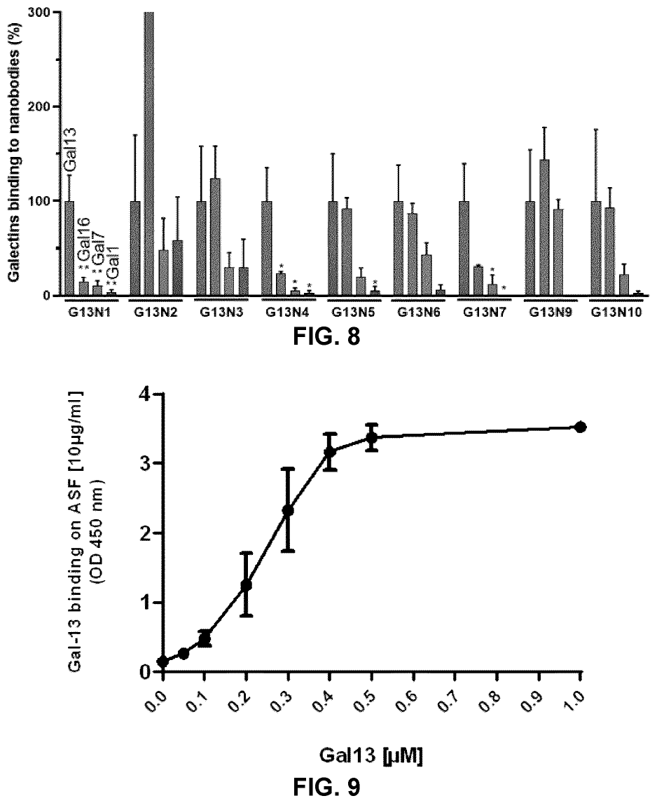

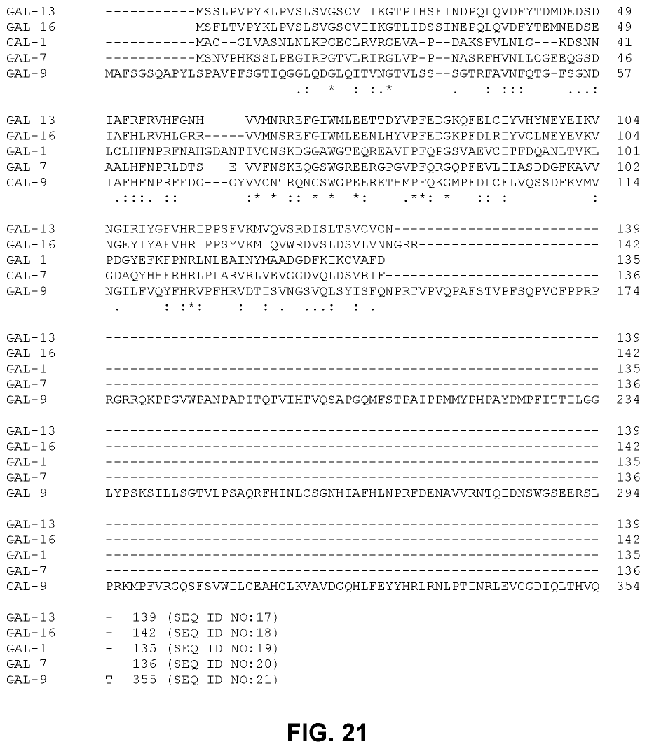

The binding of the sdAbs to various galectins (GAL-13, GAL-16, GAL-7 and GAL-1) was next tested. As shown in FIG. 8, sdAbs G13N1, G13N4 and G13N7 bind more specifically to GAL-13 than to the other Galectins. Several sdAbs were shown to bind to GAL-16 which has high sequence homology (73%) with Gal-13 (FIG. 21).

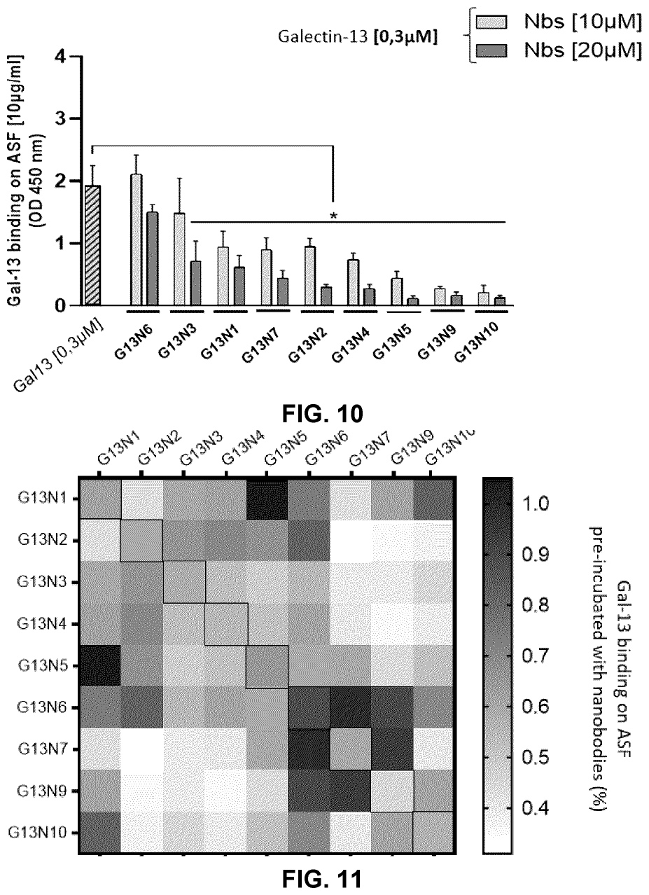

The glycoprotein asialoetuin (ASF) is a galectin ligand, including Gal-13. Recombinant human GAL-13 was shown to be able to bind to ASF in a dose-responsive manner, demonstrating that the protein GBS (Glycan Binding Site) is functional (FIG. 9). It was next tested whether the sdAbs can decrease the binding of recombinant GAL-13 to ASF. As shown in FIG. 10, at a concentration of 20 μM, all tested sdAbs were able to reduce the binding of GAL-13 to ASF, with clones G13N1, G13N2, G13N3, G13N4, G13N5, G13N7, G13N9 and G13N10 reaching statistical significance. sdAbs G13N1, G13N2, G13N4, G13N5, G13N7, G13N9 and G13N10 could also reduce the binding of GAL-13 to ASF at a concentration of 10 μM. The sdAb mixing pairing demonstrates that some mixtures, including G13N1/G13N7, G13N2/G13N7, G13N4/G13N9, are able to inhibit GAL-13 binding to ASF synergistically (FIG. 11).

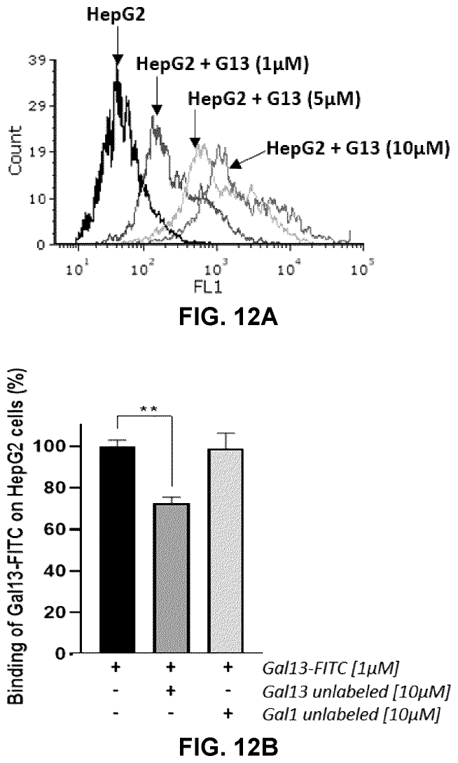

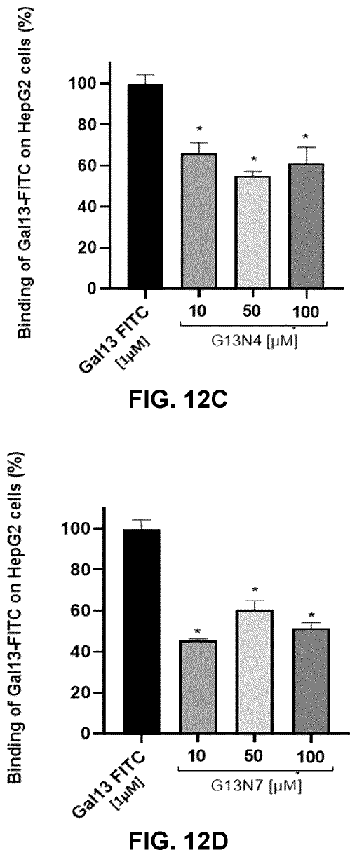

The abnormal expression of galectins is known to be linked to cancer development, progression and metastasis. FIG. 12A shows the dose-dependent binding of GAL-13-FITC on HepG2 human liver cancer cell line, which was inhibited by incubation with unlabeled GAL-13, but not unlabeled GAL-1, demonstrating a GAL-13 specific binding (FIG. 12B). Pre-incubation of GAL-13-FITC with increasing doses of G13N4 (FIG. 12C) and G13N7 (FIG. 12D) resulted in a 50% decrease of GAL-13 FITC binding on HepG2 cells.

### Example 4: Generation and Production of Galectin-9-Specific VHH Clones

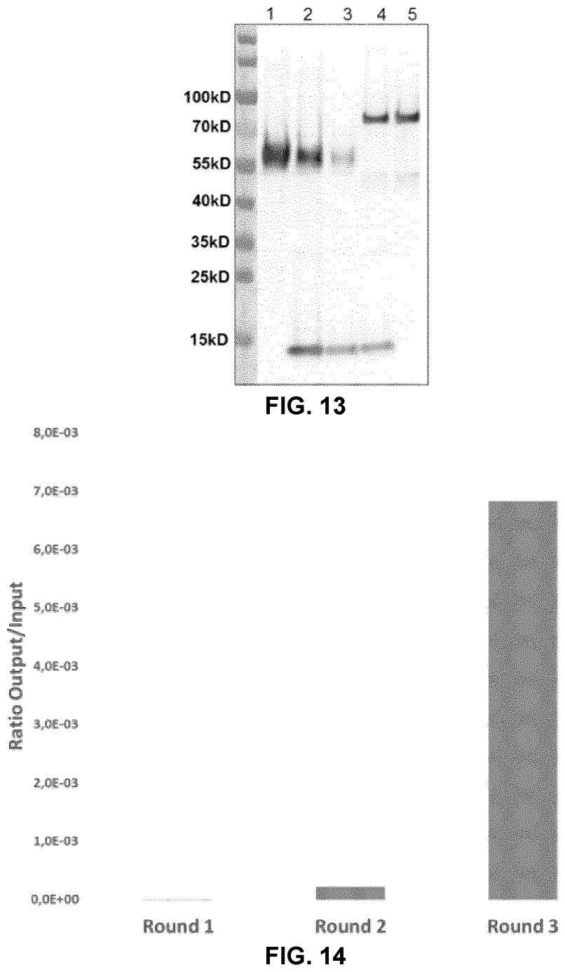

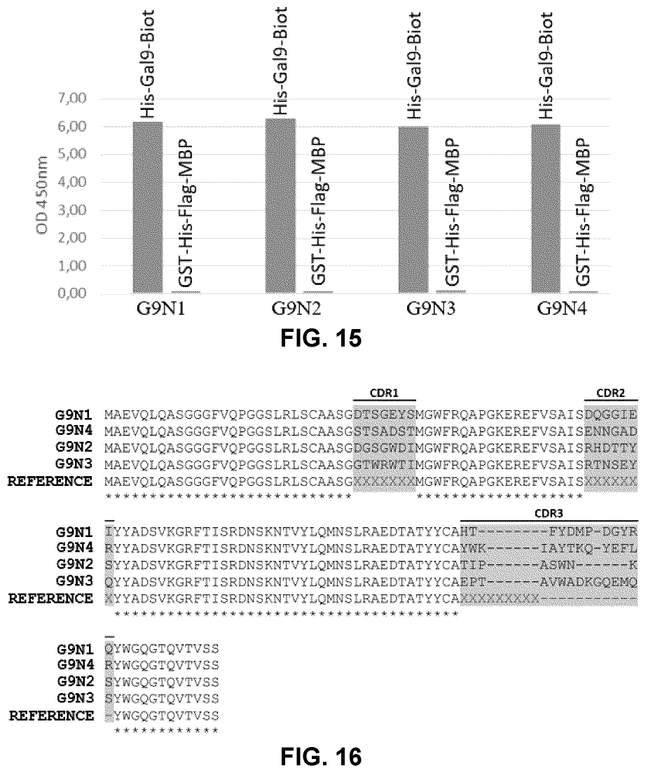

Following an initial pre-screen round of phage display carried out with GST-biotin to deplete the library from unspecific binders, unbound VHHs were incubated with beads conjugated to GAL-9-Biotin (FIG. 13). A total of three rounds of phage panning were performed. Progressive enrichment during the cycle was validated by measuring the output/input ratio (FIG. 14). A total of 90 VHHs were then picked and analyzed by ELISA for GAL-9 binders. Four (4) clones were shown to exhibit strong binding to GAL-9 (G9N1 to G9N4) (FIG. 15). The amino acid sequences of the 4 GAL-9 binding clones are shown in FIG. 16.

### Example 5: Characterization of Galectin-9-Specific sdAbs

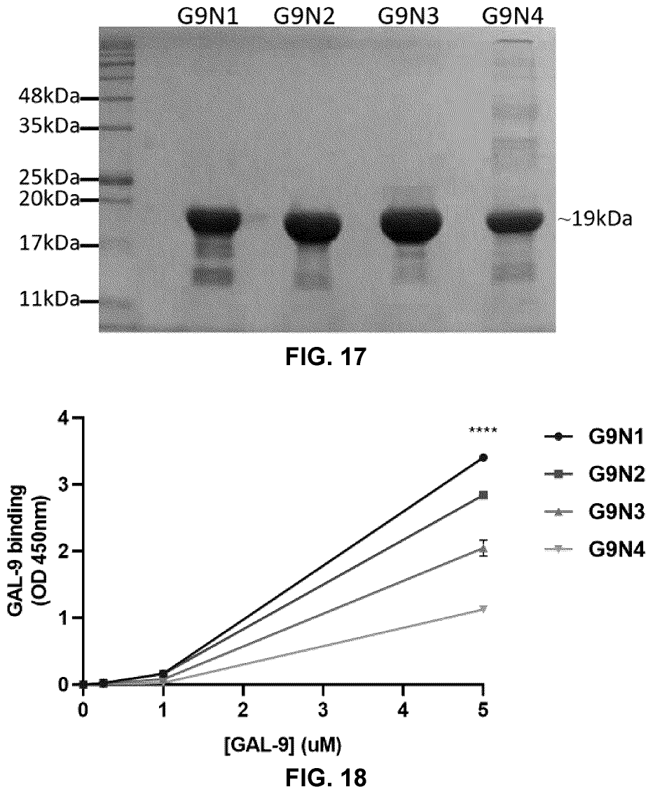

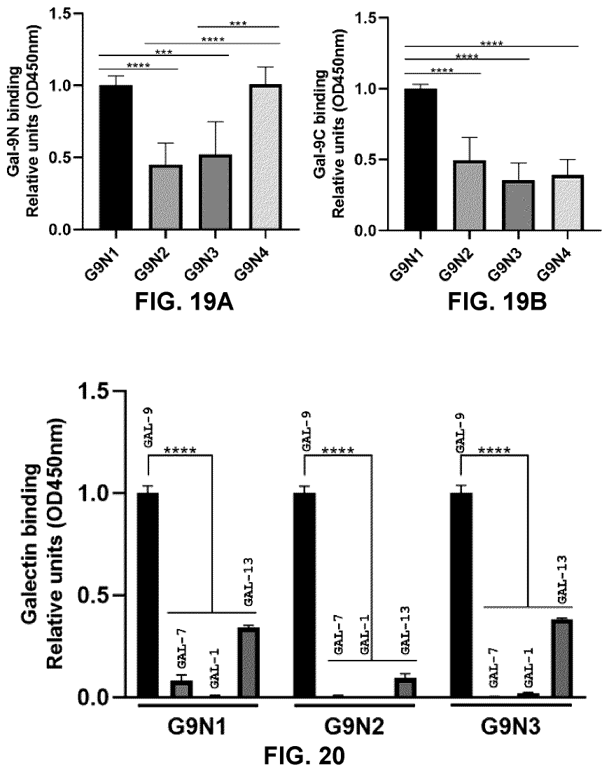

The clones were purified by conventional metal ion affinity chromatography for characterization. SDS-PAGE analyses of the clones are shown in FIG. 17. The sdAbs exhibited binding affinity for recombinant GAL-9. Significant differences in binding affinities can be observed at 5 μM of GAL-9, whereby G9N1>G9N2>G9N3>G9N4. The binding of the Gal-9-binding sdAbs to recombinant N- and C-terminal domains of human GAL-9 (GAL-9N and GAL-9C) was assessed. G9N1 and G9N4 exhibited a significantly greater affinity to Gal-9N relative to G9N2 and G9N3 (G9N1, G9N4>G9N2, G9N3) (FIG. 19A), and G9N1 exhibited a significantly greater affinity to GAL-9C relative to all other sdAbs (G9N1>G9N2, G9N3, G9N4) (FIG. 19B).

The binding of the sdAbs to various galectins (GAL-9, GAL-1, GAL-7 and GAL-13) was next tested. As shown in FIG. 20, all tested sdAbs (G9N1, G9N2 and G9N3) bind more specifically to GAL-9 than to the other Galectins. Low-affinity binding of the sdAbs to GAL-13 was also detected.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

Allo V C, Hauk V, Sarbia N, Pinto N A, Croci D O, Dalotto-Moreno T, Morales R M, Gatto S G, Cocco M N, Stupirski J C, Deladoey Á. Suppression of age-related salivary gland autoimmunity by glycosylation-dependent galectin-1-driven immune inhibitory circuits. Proceedings of the National Academy of Sciences. 2020 Mar. 24; 117 (12): 6630-9.
Barondes S H, Castronovo V, Cooper D N, Cummings R D, Drickamer K, Feizi T, Gitt M A, Hirabayashi J, Hughes C, Kasai K I. Galectins: a family of animal beta-galactoside-binding lectins. Cell. 1994; 76 (4): 597-8.
Batzke K, Büchel G, Hansen W, Schramm A. TrkB-target galectin-1 impairs immune activation and radiation responses in neuroblastoma: implications for tumour therapy. International journal of molecular sciences. 2018 March; 19 (3): 718.
Blanchard H, Bum-Erdene K, Bohari M H, Yu X. Galectin-1 inhibitors and their potential therapeutic applications: a patent review. Expert opinion on therapeutic patents. 2016 May 3; 26 (5): 537-54.
Blanchetot C, Verzijl D, Mujić-Delić A, Bosch L, Rem L, et al. 2013. Neutralizing nanobodies targeting diverse chemokines effectively inhibit chemokine function. J Biol Chem 288, 25173.
Cedeno-Laurent F, Opperman M, Barthel S R, Kuchroo V K, Dimitroff C J. Galectin-1 triggers an immunoregulatory signature in Th cells functionally defined by IL-10 expression. The Journal of Immunology. 2012 Apr. 1; 188 (7): 3127-37.
Chakraborty A, Dimitroff C J. Cancer immunotherapy needs to learn how to stick to its guns. The Journal of clinical investigation. 2019 Dec. 2; 129 (12): 5089-91.
Chen Q, Han B, Meng X, Duan C, Yang C, Wu Z, Magafurov D, Zhao S, Safin S, Jiang C, Cai J. Immunogenomic analysis reveals LGALS1 contributes to the immune heterogeneity and immunosuppression in glioma. International journal of cancer. 2019 Jul. 15; 145 (2): 517-30.
Cruz E, Kayser V. Monoclonal antibody therapy of solid tumors: clinical limitations and novel strategies to enhance treatment efficacy. Biologics: targets & therapy. 2019; 13:33.
Cummings R D, Liu F T. Galectins. Essentials of Glycobiology. 2nd edition. 2009.
Dalotto-Moreno T, Croci D O, Cerliani J P, Martinez-Allo V C, Dergan-Dylon S, Méndez-Huergo S P, Stupirski J C, Mazal D, Osinaga E, Toscano M A, Sundblad V. Targeting galectin-1 overcomes breast cancer-associated immunosuppression and prevents metastatic disease. Cancer research. 2013 Feb. 1; 73 (3): 1107-17.
   De Genst E, Silence K, Decanniere K, Conrath K, Loris R, Kinne J, Muyldermans S, Wyns L. 2006. Molecular basis for the preferential cleft recognition by dromedary heavy-chain antibodies. Proc Nat Acad Sci 103, 4586.
Grosset A A, Labrie M, Vladoiu M C, Yousef E M, Gaboury L, St-Pierre Y. Galectin signatures contribute to the heterogeneity of breast cancer and provide new prognostic information and therapeutic targets. Oncotarget. 2016 Apr. 5; 7 (14): 18183.

Hamers-Casterman C T, Atarhouch T, Muyldermans S, Robinson G, Hammers C, Songa E B, Bendahman N, Hammers R. Naturally occurring antibodies devoid of light chains. Nature. 1993 June; 363 (6428): 446-8.
Jin Q Y, Li Y S, Qiao X H, Yang J W, Guo X L. Targeting galectins in T cell-based immunotherapy within tumor microenvironment. Life Sciences. 2021 Mar. 27:119426.
Labrie M, De Araujo L O, Communal L, Mes-Masson A M, St-Pierre Y. Tissue and plasma levels of galectins in patients with high grade serous ovarian carcinoma as new predictive biomarkers. Scientific reports. 2017 Oct. 16; 7 (1): 1-3.
Liu F T, Rabinovich G A. Galectins as modulators of tumour progression. Nature Reviews Cancer. 2005 January; 5 (1): 29-41.
Liu S D, Whiting C C, Tomassian T, Pang M, Bissel S J, Baum L G, Mossine W, Poirier F, Huflejt M E, Miceli M C. Endogenous galectin-1 enforces class I-restricted TCR functional fate decisions in thymocytes. Blood, The Journal of the American Society of Hematology. 2008 Jul. 1; 112 (1): 120-30.
Lykken J M, Horikawa M, Minard-Colin V, Kamata M, Miyagaki T, Poe J C, Tedder T F. Galectin-1 drives lymphoma CD20 immunotherapy resistance: validation of a preclinical system to identify resistance mechanisms. Blood, The Journal of the American Society of Hematology. 2016 Apr. 14; 127 (15): 1886-95
McMahon C, Baier A S, Pascolutti R, Wegrecki M, Zheng S, et al. 2018. Yeast surface display platform for rapid discovery of conformationally selective nanobodies. Nat Struct Mol Biol 25, 289
Moutel S, Bery N, Bernard V, Keller L, Lemesre E, et al. 2016. NaLi-H1: A universal synthetic library of humanized nanobodies providing highly functional antibodies and intrabodies. Elife 5.
Nambiar D K, Aguilera T, Cao H, Kwok S, Kong C, Bloomstein J, Wang Z, Rangan V S, Jiang D, Von Eyben R, Liang R. Galectin-1-driven T cell exclusion in the tumor endothelium promotes immunotherapy resistance. The Journal of clinical investigation. 2019 Dec. 2; 129 (12): 5553-67.
Perillo N L, Uittenbogaart C H, Nguyen J T, Baum L G. Galectin-1, an endogenous lectin produced by thymic epithelial cells, induces apoptosis of human thymocytes. The Journal of experimental medicine. 1997 May 19; 185 (10): 1851-8.
Pinho S S, Reis C A. Glycosylation in cancer: mechanisms and clinical implications. Nature Reviews Cancer. 2015 September; 15 (9): 540-55.
Rabinovich G A, Toscano M A. Turning ‘sweet’ on immunity: galectin-glycan interactions in immune tolerance and inflammation. Nature Reviews Immunology. 2009 May; 9 (5): 338-52.
Rodríguez E, Schetters S T, van Kooyk Y. The tumour glyco-code as a novel immune checkpoint for immunotherapy. Nature Reviews Immunology. 2018 March; 18 (3): 204-11.
Salatino M, Dalotto-Moreno T, Rabinovich G A. Thwarting galectin-induced immunosuppression in breast cancer. Oncoimmunology. 2013 May 1; 2 (5): e24077.
Sundblad V, Morosi L G, Geffner J R, Rabinovich G A. Galectin-1: a jack-of-all-trades in the resolution of acute and chronic inflammation. The Journal of Immunology. 2017 Dec. 1; 199 (11): 3721-30.
Tang D, Gao J, Wang S, Yuan Z, Ye N, Chong Y, Xu C, Jiang X, Li B, Yin W, Miao Y. Apoptosis and anergy of T cell induced by pancreatic stellate cells-derived galectin-1 in pancreatic cancer. Tumor Biology. 2015 July; 36 (7): 5617-26.
Than N G, Balogh A, Romero R, Kárpáti É, Erez O, Szilágyi A, Kovalszky I, Sammar M, Gizurarson S, Matkó J, Závodszky P. Placental protein 13 (PP13)—a placental immunoregulatory galectin protecting pregnancy. Frontiers in immunology. 2014 Aug. 20; 5:348.
Van Woensel M, Mathivet T, Wauthoz N, Rosière R, Garg A D, Agostinis P, Mathieu V, Kiss R, Lefranc F, Boon L, Belmans J. Sensitization of glioblastoma tumor micro-environment to chemo- and immunotherapy by Galectin-1 intranasal knock-down strategy. Scientific reports. 2017 Apr. 27; 7 (1): 1-4.
Verschuere T, Toelen J, Maes W, Poirier F, Boon L, Tousseyn T, Mathivet T, Gerhardt H, Mathieu V, Kiss R, Lefranc F. Glioma-derived galectin-1 regulates innate and adaptive antitumor immunity. International journal of cancer. 2014 February; 134 (4): 873-84.
Wu A A, Drake V, Huang H S, Chiu S, Zheng L. Reprogramming the tumor microenvironment: tumor-induced immunosuppressive factors paralyze T cells. Oncoimmunology. 2015 Jul. 3; 4 (7): e1016700.
Salema et al., MAbs vol. 8, No. 7, 1286-1301.
Gene, R W et al., (2015) J Immunol Methods 416, 29-39.

### Kumaran, J. (2012). Methods Mol Biol 911, 105-124.

Cousin and Cloninger, Int J Mol Sci. 2016 September; 17 (9): 1566.
Sundblad et al., J Immunol Dec. 1, 2017, 199 (11) 3721-3730.
Seki M, et al., Arthritis Rheum. 2007; 56 (12): 3968-76.
O'Brien et al., Arthritis Research & Therapy volume 20, Article number: 31 (2018).

We claim:

1. A monovalent antibody that binds to human galectin-13 (GAL-13), wherein the monovalent antibody comprises one of the following combinations of complementarity determining regions (CDRs):
a CDR1 comprising or consisting of the amino acid sequence of SEQ ID NO:22; a CDR2 comprising or consisting of the amino acid sequence of SEQ ID NO: 23; and a CDR3 comprising or consisting of the amino acid sequence of SEQ ID NO:24;
a CDR1 comprising or consisting of the amino acid sequence of SEQ ID NO:25; a CDR2 comprising or consisting of the amino acid sequence of SEQ ID NO:26; and a CDR3 comprising or consisting of the amino acid sequence of SEQ ID NO:27;
a CDR1 comprising or consisting of the amino acid sequence of SEQ ID NO:28; a CDR2 comprising or consisting of the amino acid sequence of SEQ ID NO: 29; and a CDR3 comprising or consisting of an amino acid sequence having at least 70% identity with the sequence of SEQ ID NO:30;
a CDR1 comprising or consisting of the amino acid sequence of SEQ ID NO:31; a CDR2 comprising or consisting of the amino acid sequence of SEQ ID NO: 32; and a CDR3 comprising or consisting of the amino acid sequence of SEQ ID NO:33;
a CDR1 comprising or consisting of the amino acid sequence of SEQ ID NO:34; a CDR2 comprising or consisting of the amino acid sequence of SEQ ID NO: 35; and a CDR3 comprising or consisting of the amino acid sequence of SEQ ID NO:36;
a CDR1 comprising or consisting of the amino acid sequence of SEQ ID NO:37; a CDR2 comprising or consisting of the amino acid sequence of SEQ ID NO: 38; and a CDR3 comprising or consisting of the amino acid sequence of SEQ ID NO:39;
a CDR1 comprising or consisting of the amino acid sequence of SEQ ID NO:40; a CDR2 comprising or consisting of the amino acid sequence of SEQ ID NO: 41; and a CDR3 comprising or consisting of the amino acid sequence of SEQ ID NO:42;
a CDR1 comprising or consisting of the amino acid sequence of SEQ ID NO:43; a CDR2 comprising or consisting of the amino acid sequence of SEQ ID NO: 44; and a CDR3 comprising or consisting of the amino acid sequence of SEQ ID NO:45;
a CDR1 comprising or consisting of the amino acid sequence of SEQ ID NO:46; a CDR2 comprising or consisting of the amino acid of SEQ ID NO: 47; and a CDR3 comprising or consisting of the amino acid sequence of SEQ ID NO:48; or
a CDR1 comprising or consisting of the amino acid sequence of SEQ ID NO:49; a CDR2 comprising or consisting of the amino acid sequence of SEQ ID NO: 50; and a CDR3 comprising or consisting of the amino acid sequence of SEQ ID NO:51.

2-12. (canceled)

13. The monovalent antibody of claim 1, wherein the monovalent antibody is a single-domain antibody.

14-15. (canceled)

16. The monovalent antibody of claim 1, which comprises: (i) a FR1 comprising the sequence of SEQ ID NO:52 or SEQ ID NO:68;
(ii) a FR2 comprising the sequence of SEQ ID NO:53;
(iii) a FR3 comprising the sequence of SEQ ID NO:54;
(iv) a FR4 comprising the sequence of SEQ ID NO:55 or SEQ ID NO:69; or
(v) any combination of (i) to (iv).

17-18. (canceled)

19. The monovalent antibody of claim 1, comprising one of the amino acid sequences set forth in SEQ ID NOs: 1-10.

20. The monovalent antibody according to claim 1, wherein said monovalent antibody further binds to GAL-16.

21. The monovalent antibody according to claim 1, wherein said monovalent antibody is fused to at least one antibody constant domain, or a fragment thereof.

22. (canceled)

23. The monovalent antibody of claim 1, wherein said antibody is conjugated to a label, a nanoparticle, a drug, a peptide, a nucleic acid, a toxin, an enzyme, a radioisotope, a half-life extending moiety, or a targeting agent.

24. A nucleic acid comprising a nucleotide sequence encoding the monovalent antibody defined in claim 1.

25-29. (canceled)

30. A method for binding human galectin-13 (GAL-13) and/or GAL-16 comprising contacting said GAL-13 and/or GAL-16 with the monovalent antibody of claim 1.

31-34. (canceled)

35. A method for treating a galectin-13 (GAL-13)- and/or galectin-16-expressing cancer in a subject, said method comprising administering to said subject an effective amount of the monovalent antibody of claim 1, a nucleic acid comprising a nucleotide sequence encoding said monovalent antibody, or a pharmaceutical composition comprising said monovalent antibody or nucleic acid.

36-57. (canceled)

58. A monovalent antibody that binds to human galectin-9 (GAL-9), wherein the monovalent antibody comprises one of the following combinations of complementarity determining regions (CDRs):
a CDR1 comprising or consisting of the amino acid sequence DTSGEYS (SEQ ID NO:56); a CDR2 comprising or consisting of the amino acid sequence DQGGIEI (SEQ ID NO:57); and a CDR3 comprising or consisting of the amino acid sequence HTFYDMPDGYRQ (SEQ ID NO:58);
a CDR1 comprising or consisting of the amino acid sequence STSADST (SEQ ID NO:59); a CDR2 comprising or consisting of the amino acid sequence ENNGADR (SEQ ID NO:60); and a CDR3 comprising or consisting of the amino acid sequence YWKIAYTKQYEFL (SEQ ID NO: 61);
a CDR1 comprising or consisting of the amino acid sequence DGSGWDI (SEQ ID NO:62); a CDR2 comprising or consisting of the amino acid sequence RHDTTYS (SEQ ID NO:63); and a CDR3 comprising or consisting of the amino acid sequence TIPASWNKS (SEQ ID NO:64); or
a CDR1 comprising or consisting of the amino acid sequence GTWRWTI (SEQ ID NO:65); a CDR2 comprising or consisting of the amino acid sequence RTNSEYQ (SEQ ID NO:66); and a CDR3 comprising or consisting of the amino acid sequence EPTAVWADKGQEMQS (SEQ ID NO:67).

59-63. (canceled)

64. The monovalent antibody of claim 58, wherein the monovalent antibody is a single-domain antibody.

65-66. (canceled)

67. The monovalent antibody of claim 58, which comprises:
(i) a FR1 comprising the sequence of SEQ ID NO:52;
(ii) a FR2 comprising the sequence of SEQ ID NO:53;
(iii) a FR3 comprising the sequence of SEQ ID NO:54;
(iv) a FR4 comprising the sequence of SEQ ID NO:55; or
(v) any combination of (i) to (iv).

68-69. (canceled)

70. The monovalent antibody of claim 58, comprising one of the amino acid sequences set forth in SEQ ID NOs: 12-15.

71. The monovalent antibody according to claim 58, wherein said monovalent antibody is fused to at least one antibody constant domain, or a fragment thereof.

72-73. (canceled)

74. The monovalent antibody of claim 58, wherein said antibody is conjugated to a label, a nanoparticle, a drug, a peptide, a nucleic acid, a toxin, an enzyme, a radioisotope, or a half-life extending moiety.

75. A nucleic acid comprising a nucleotide sequence encoding the monovalent antibody defined in claim 58.

76-80. (canceled)

81. A method for binding human galectin-9 (GAL-9) comprising contacting said GAL-9 with the monovalent antibody of claim 58.

82. A method for treating a galectin-9 (GAL-9)-expressing cancer in a subject, said method comprising administering to said subject an effective amount of the monovalent antibody of claim 58, a nucleic acid comprising a nucleotide sequence encoding said monovalent antibody, or a composition comprising said monovalent antibody or nucleic acid.

83-90. (canceled)

91. A method for treating an inflammatory disease associated with GAL-9 activity, a disease or condition associated with pathological neovascularization or angiogenesis, in a subject, said method comprising administering an effective amount of the monovalent antibody of claim 58, a nucleic acid comprising a nucleotide sequence encoding said monovalent antibody, or a composition comprising said monovalent antibody or nucleic acid.

92-118. (canceled)


SEQUENCE LISTING (71 sequences)

SEQ ID NO: 1 - protein, 121 aa, synthetic construct
MAEVQLQASGGGFVQPGGSLRLSCAASGFGSNRSTMGWFRQAPGKEREFVSAISSLSSDPPYYADSVKGRFTISRDNSKNTVYLQMNSLRAEDTATYYCAPAGMRGVMEYWGQGTQVTVSS

SEQ ID NO: 2 - protein, 127 aa, synthetic construct
MAEVQLQASGGGFVQPGGSLRLSCAASGSTSRSSGMGWFRQAPGKEREFVSAISYRGSGARYYADSVKGRFTISRDNSKNTVYLQMNSLRAEDTATYYCAMWQRQIQPGTRPVWKYWGQGTQVTVSS

SEQ ID NO: 3 - protein, 124 aa, synthetic construct
MAEVQLQASGGGFVQPGGSLRLSCAASGRGSRWYGMGWFRQAPGKEREFVSAISAQHSTRAYYADSVKGRFTISRDNSKNTVYLQMNSLRAEDTATYYCAGYMSKMGERKWKYWGQGTQVTVSS

SEQ ID NO: 4 - protein, 121 aa, synthetic construct
MAEVQLQASGGGFVQPGGSLRLSCAASGSSYAGSAMGWFRQAPGKEREFVSAISSDPDTKAYYADSVKGRFTISRDNSKNTVYLQMNSLRAEDTATYYCAISGQYPYARYWGHGTQVTVSS

SEQ ID NO: 5 - protein, 130 aa, synthetic construct
MAEVQLQASGGGFVQPGGSLRLSCAASGYTSNEDSMGWFRQAPGKEREFVSAISRGPSFRTYYADSVKGRFTISRDNSKNTVYLQMNSLRAEDTATYYCAIRLGRLYRYGDLTPKSAYYWGQGTQVTVSS

SEQ ID NO: 6 - protein, 121 aa, synthetic construct
MAEVQLQASGGGFVQPGGSLRLSCAASGRTSSLTSMGWFRQAPGKEREFVSAISFSGSMFTYYADSVKGRFTISRDNSKNTVYLQMNSLRAEDTATYYCADKWMHKPANYWGQGTQVTVSS

SEQ ID NO: 7 - skipped

SEQ ID NO: 8 - protein, 127 aa, synthetic construct
MAEVQLQASGGGFVQPGGSLRLSCAASGTTWQETKMGWFRQAPGKEREFVSAISRGPNYYPYYADSVKGRFTISRDNSKNTVYLQMNSLRAEDTATYYCADEWMGSINARGHSGYYWGQGTQVTVSS

SEQ ID NO: 9 - protein, 124 aa, synthetic construct
MAEVQLQASGGGFVQPGGSLRLSCAASGRGSALTTMGWFRQAPGKEREFVSAISGDTGLYVYYADSVKGRFTISRDNSKNTVYLQMNSLRAEDTATYYCAGKWLGGRQADTRYWGQGTQVTVSS

SEQ ID NO: 10 - protein, 126 aa, synthetic construct
MAEVQLQASGGGFVQPGGSLRLSCAASGRTSNQTIGWFRQAPGKEREFVSAISARFDEYGYYADSVKGRFTISRDNSKNTVYLQMNSLRAEDTATYYCALGWPQKSTWRAYTAEYWGQGTQVTVSS

SEQ ID NO: 11 - protein, 121 aa, synthetic construct
MAEVQLQASGGGFVQPGGSLRLSCAASGXXXXXXXMGWFRQAPGKEREFVSAISXXXXXXXYYADSVKGRFTISRDNSKNTVYLQMNSLRAEDTATYYCAXXXXXXXXXYWGQGTQVTVSS

SEQ ID NO: 12 - protein, 124 aa, synthetic construct
MAEVQLQASGGGFVQPGGSLRLSCAASGDTSGEYSMGWFRQAPGKEREFVSAISDQGGIEIYYADSVKGRFTISRDNSKNTVYLQMNSLRAEDTATYYCAHTFYDMPDGYRQYWGQGTQVTVSS

SEQ ID NO: 13 - protein, 126 aa, synthetic construct
MAEVQLQASGGGFVQPGGSLRLSCAASGSTSADSTMGWFRQAPGKEREFVSAISENNGADRYYADSVKGRFTISRDNSKNTVYLQMNSLRAEDTATYYCAYWKIAYTKQYEFLRYWGQGTQVTVSS

SEQ ID NO: 14 - protein, 121 aa, synthetic construct
MAEVQLQASGGGFVQPGGSLRLSCAASGDGSGWDIMGWFRQAPGKEREFVSAISRHDTTYSYYADSVKGRFTISRDNSKNTVYLQMNSLRAEDTATYYCATIPASWNKSYWGQGTQVTVSS

SEQ ID NO: 15 - protein, 127 aa, synthetic construct
MAEVQLQASGGGFVQPGGSLRLSCAASGGTWRWTIMGWFRQAPGKEREFVSAISRTNSEYQYYADSVKGRFTISRDNSKNTVYLQMNSLRAEDTATYYCAEPTAVWADKGQEMQSYWGQGTQVTVSS

SEQ ID NO: 16 - protein, 122 aa, synthetic construct
MAEVQLQASGGGFVQPGGSLRLSCAASGXXXXXXXMGWFRQAPGKEREFVSAISXXXXXXXYYADSVKGRFTISRDNSKNTVYLQMNSLRAEDTATYYCAXXXXXXXXXXYWGQGTQVTVSS

SEQ ID NO: 17 - protein, 139 aa, Homo sapiens
MSSLPVPYKLPVSLSVGSCVIIKGTPIHSFINDPQLQVDFYTDMDEDSDIAFRFRVHFGNHVVMNRREFGIWMLEETTDYVPFEDGKQFELCIYVHYNEYEIKVNGIRIYGFVHRIPPSFVKMVQVSRDISLTSVCVCN

SEQ ID NO: 18 - protein, 142 aa, Homo sapiens
MSFLTVPYKLPVSLSVGSCVIIKGTLIDSSINEPQLQVDFYTEMNEDSEIAFHLRVHLGRRVVMNSREFGIWMLEENLHYVPFEDGKPFDLRIYVCLNEYEVKVNGEYIYAFVHRIPPSYVKMIQVWRDVSLDSVLVNNGRR

SEQ ID NO: 19 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 20 - protein, 136 aa, Homo sapiens
MSNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

SEQ ID NO: 21 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 22 - protein, 7 aa, synthetic construct
FGSNRST

SEQ ID NO: 23 - protein, 7 aa, synthetic construct
SLSSDPP

SEQ ID NO: 24 - protein, 9 aa, synthetic construct
PAGMRGVME

SEQ ID NO: 25 - protein, 7 aa, synthetic construct
STSRSSG

SEQ ID NO: 26 - protein, 7 aa, synthetic construct
YRGSGAR

SEQ ID NO: 27 - protein, 15 aa, synthetic construct
MWQRQIQPGTRPVMK

SEQ ID NO: 28 - protein, 7 aa, synthetic construct
RGSRWYG

SEQ ID NO: 29 - protein, 7 aa, synthetic construct
AQHSTRA

SEQ ID NO: 30 - protein, 12 aa, synthetic construct
GYMSKMGERKWK

SEQ ID NO: 31 - protein, 7 aa, synthetic construct
SSYAGSA

SEQ ID NO: 32 - protein, 7 aa, synthetic construct
SDPDTKA

SEQ ID NO: 33 - protein, 9 aa, synthetic construct
ISGQYPYAR

SEQ ID NO: 34 - protein, 7 aa, synthetic construct
YTSNEDS

SEQ ID NO: 35 - protein, 7 aa, synthetic construct
RGPSFRT

SEQ ID NO: 36 - protein, 18 aa, synthetic construct
IRLGRLYRYGDLTPKSAY

SEQ ID NO: 37 - protein, 7 aa, synthetic construct
RTSSLTS

SEQ ID NO: 38 - protein, 7 aa, synthetic construct
FSGSMFT

SEQ ID NO: 39 - protein, 9 aa, synthetic construct
DKWMHKPAN

SEQ ID NO: 40 - protein, 7 aa, synthetic construct
RTWNSYT

SEQ ID NO: 41 - protein, 7 aa, synthetic construct
GEPGGIR

SEQ ID NO: 42 - protein, 9 aa, synthetic construct
DRWPNKHDR

SEQ ID NO: 43 - protein, 7 aa, synthetic construct
TTWKQET

SEQ ID NO: 44 - protein, 7 aa, synthetic construct
RGPNYYP

SEQ ID NO: 45 - protein, 15 aa, synthetic construct
DEWMGSINARHGSGY

SEQ ID NO: 46 - protein, 7 aa, synthetic construct
RGSALTT

SEQ ID NO: 47 - protein, 7 aa, synthetic construct
GDTGLYV

SEQ ID NO: 48 - protein, 12 aa, synthetic construct
GKWLGGRQADTR

SEQ ID NO: 49 - protein, 6 aa, synthetic construct
RTSNQT

SEQ ID NO: 50 - protein, 7 aa, synthetic construct
ARFDEYG

SEQ ID NO: 51 - protein, 15 aa, synthetic construct
LGWPQKSTWRAYTAE

SEQ ID NO: 52 - protein, 28 aa, synthetic construct
MAEVQLQASGGGFVQPGGSLRLSCAASG

SEQ ID NO: 53 - protein, 19 aa, synthetic construct
MGWFRQAPGKEREFVSAIS

SEQ ID NO: 54 - protein, 39 aa, synthetic construct
YYADSVKGRFTISRDNSKNTVYLQMNSLRAEDTATYYCA

SEQ ID NO: 55 - protein, 12 aa, synthetic construct
YWGQGTQVTVSS

SEQ ID NO: 56 - protein, 7 aa, synthetic construct
DTSGEYS

SEQ ID NO: 57 - protein, 7 aa, synthetic construct
DQGGIEI

SEQ ID NO: 58 - protein, 12 aa, synthetic construct
HTFYDMPDGYRQ

SEQ ID NO: 59 - protein, 7 aa, synthetic construct
STSADST

SEQ ID NO: 60 - protein, 7 aa, synthetic construct
ENNGADR

SEQ ID NO: 61 - protein, 13 aa, synthetic construct
YWKIAYTKQYEFL

SEQ ID NO: 62 - protein, 7 aa, synthetic construct
DGSGWDI

SEQ ID NO: 63 - protein, 7 aa, synthetic construct
RHDTTYS

SEQ ID NO: 64 - protein, 9 aa, synthetic construct
TIPASWNKS

SEQ ID NO: 65 - protein, 7 aa, synthetic construct
GTWRWTI

SEQ ID NO: 66 - protein, 7 aa, synthetic construct
RTNSEYQ

SEQ ID NO: 67 - protein, 15 aa, synthetic construct
EPTAVWADKGQEMQS

SEQ ID NO: 68 - protein, 28 aa, synthetic construct
IAEVQLQASGGGFVQPGGSLRLSCAASG

SEQ ID NO: 69 - protein, 12 aa, synthetic construct
YWGHGTQVTVSS

SEQ ID NO: 70 - protein, 148 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQ

SEQ ID NO: 71 - protein, 146 aa, Homo sapiens
TPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT